(19) United States
(12) Patent Application Publication
(10) Pub. No.: US 2026/0227409 A1
(43) Pub. Date: Aug. 6, 2026

(54) DISTINCTIVE IDENTIFICATION AND USE OF A LIQUID BIOPSY CANCER BIOMARKER FOR CANCER DETECTION, MANAGEMENT AND TREATMENT

(71) Applicant: RARECELLS INC., Paris Cedex 06 (FR)

(72) Inventor: Patrizia PATERLINI, Paris (FR)

(73) Assignee: RARECELLS INC., Paris Cedex 06 (FR)

(21) Appl. No.: 19/147,796

(22) PCT Filed: Jan. 17, 2024
(86) PCT No.: PCT/IB2024/000032

§ 371 Date: Jul. 14, 2025

Related U.S. Application Data
(60) Provisional application No. 63/480,220, filed on Jan. 17, 2023.

(51) Int. Cl. G01N 33/575; G01N 33/50

(52) U.S. Cl. CPC G01N 33/5759; G01N 33/5091; G01N 2333/9128

(57) ABSTRACT

Methods for the distinctive identification and characterization of Cancer Giant Cells, which have a size of 15 microns or more (major axis), which express abnormally high amounts of telomerase, and/or which express syncytin and/or cancer associated antigens and Giant Cells associated antigens such as macrophage antigens which distinguish them from non-cancer Giant Cells. Use of Cancer Giant Cells as a liquid biopsy biomarker to determine the presence of cancer, early detection of cancer, early detection of cancer recurrence, determination of therapy and of treatment response. Use of the Cancer Giant Cells liquid biopsy biomarker for determining the risk of having cancer or cancer recurrence or of being at risk of developing cancer or cancer recurrence, and for treating the subject with personalized therapy against cancer to treat or prevent cancer or cancer recurrence. A kit for detecting Cancer Giant Cells and its use to detect cancer and cancer recurrence and manage cancer treatment is disclosed.

### BACKGROUND OF THE INVENTION

Field of the Invention The present invention pertains to the fields of diagnostic and therapeutic oncology. More specifically, it relates to use of Giant Cells (GC), distinctively identified as Cancer Giant Cells or Circulating Cancer Giant Cells, expressing telomerase, syncytins or other cancer-specific biomarkers for diagnosis, management and treatment of cancer.

Description of Related Art. Cancer is the second leading cause of death in most countries, including in the United States. Cancer mortality is strictly linked to how and when cancer is diagnosed and early cancer detection has been proven to reduce mortality. As a matter of fact, a decrease in cancer mortality of 45% for colon cancer and 41 to 92% for cervical cancer has been registered in the last decades and attributed to the implementation of effective cancer screening programs. These successful screening programs led to the identification of precursor lesions (e.g., cervical intraepithelial neoplasia (CIN) with cervical cancer screening and colonic polyps with colorectal cancer screening) where the treatment of the precursor lesion leads to a decrease in the incidence of invasive cancer over time. However, a key feature of colon and cervical cancer screening is the ability to directly access the tissue of interest, the cancer cells, and apply an adequate screening test.

Detection and removal of pre-cancer or early cancer in other cancer types has not been as successful. Accessing cancer cells derived from other cancer types at an early stage of cancer development is extremely challenging. Patients with very early-stage cancers are mostly asymptomatic, and thus do not undergo medical imaging analyses. Furthermore, imaging approaches have a limited sensitivity.

It is considered that, in general, by the time a tumor becomes detectable, it has achieved a mass of approximately 1 g or 109 tumor cells. This means that, at this stage, anti-cancer treatments cannot in general eliminate the totality of tumor cells.

Early diffusion of tumor cells in blood was first observed in animal models and is reviewed in Laget et al., Technical insights into highly sensitive isolation and molecular characterization of fixed and live circulating tumor cells for early detection of tumor invasion, PLOS ONE, 2017, 12(1): e0169427). On average, about one out of 1000 cancer cells from the tumor mass is thought to be able to invade the blood compartment. These data are consistent with earlier work using animal models of fibrosarcoma and breast cancer.

Laget et al. considered that, by taking this value as a reference, a tumor containing 500,000 cells with a size of about 0.5 mm in diameter (~0.5 mg of tumor) would spread 500 cancer cells into the 5 liters of human blood. Such a small tumor might therefore be detected by a very sensitive collection system which LLOD is 1 tumor cell per 10 mL of blood like ISET. However, even if these calculations were an estimate and individual cancers have different invasion capabilities, they provide a reference value consistent with the possible detection of tumor cells before an invasive tumor reaches the size of a few mm in diameter which makes it detectable by imaging. At this stage, it could spread around 4,000 tumor cells in blood, equivalent to 8 tumor cells per 10 mL of blood.

Thus, a very sensitive detection of circulating cancer cells is required for early diagnosis, management and treatment of cancer through a liquid biopsy approach. Cancer Giant Cells are particularly interesting to this aim because they have been suggested to initiate tumorigenesis and tumor recurrence after therapy (Liu J, Erenpreisa J, Sikora E. Polyploid giant cancer cells: an emerging new field of cancer biology. Semin Cancer Biol. 2022; 81:1-4) and have been demonstrated in solid tumor biopsies. However, the formation of Giant Cells can also be relied to conditions of chronic inflammation and chronic infection (Brodbeck W G, Anderson J M. Giant cell formation and function. Curr Opin Hematol. 2009; 16:53-7.). According to reported studies, increase in cell size may serve as a response to environmental stresses switching proliferative mitosis to intranuclear replication. Giant cells can be formed by fusion of various cells such as macrophage, epithelioid cells, monocytes, etc. This process generates multi-nucleated, large in size cells, generally ranging from 25 to 300 μm, which are, most of the times, present at the site of chronic inflammation or as a response to infections.

The inventor has been able to observe, in blood analyses, both circulating Giant Cells in patients having infectious diseases (e.g. COVID or other infections) and in patients having cancer. If it is easy to identify cancer Giant Cells when they are found inside a tumorous tissue, it is extremely difficult to identify them when they are found in blood, especially in subjects who could have a preclinical cancer, which means a cancer at early stage, not generating signs or symptoms.

Thus, the problem to be solved is the reliable and distinctive identification of circulating Cancer Giant Cells related to the presence of a cancer in a body, from circulating Giant Cells related to chronic inflammation, chronic infection, or stress. The distinctive identification of circulating Cancer Giant Cells related to the presence of a cancer in a body allows the early detection of cancer and of cancer recurrence and the cancer management through liquid biopsy including cancer's adequate and timely treatment.

In view of the problems and limitations of conventional liquid biopsy's methods for diagnosing cancer, the inventor sought a new means for quickly and conveniently diagnosing early-stage cancer or recurrence of cancer.

The inventor considered that a new method for cancer eradication or prevention of development or recurrence of cancer should involve early cancer detection or early cancer recurrence detection, ideally the detection of the first cancer cells, which allows one to eliminate the cancer cells while they are low in number and low in heterogeneity.

These first cancer cells are considered to be Cancer Giant Cells, and they can be found and distinctively identified in body liquids (circulating Cancer Giant Cells). The inventor thus sought a method that permits the detection of such Cancer Giant Cells non-invasively such as by analysis of blood or other bodily fluids. Such a non-invasive approach would allow one to perform repeated testing thus to keep an individual under surveillance for appearance of the first Cancer Giant Cells.

The inventor proposed to identify CTC/CGC (Circulating Tumor Cells/Cancer Giant Cells) based on their expression of tumor markers and distinguish them from non-tumor cells/non-cancer giant cells.

The inventor further proposed to design and develop a personalized cancer prophylaxis or treatment, based on the CTC's/CGC's tumor markers and/or combination of tumor markers and/or combination of tumor and non-tumor markers that selectively would target the first cancer giant cells (CGC) without significantly affecting other non-cancer cells in the body.

The inventor also sought to provide a method for surveillance of new cancer cells either before cancer is conventionally detected or after an anti-cancer treatment as a companion diagnostic test to horizontally follow cancer cells elimination or their possible reappearance.

The inventor considered that a distinction of CTC/CGC from non-cancer cells be made based on their expression of telomerase, syncytin, HERV proteins, CD71, racemase, RunX2, RunX1, PSMA or other tumor specific markers that could allow not only the detection of the very early steps of cancer development or recurrence, but also the identification of selective therapeutic targeted treatments for CTC/CGC elimination without elimination of the non-cancer cells and non-cancer GC.

Other markers include RUNX2 gene encodes a transcription factor that acts as a “master switch,” regulating genes involved in the development of several tissues including bones, teeth, and cartilage. RUNX2 is also involved in tumor cells invasion and in the development of malignant tumors. Runt-related transcription factor 1 (RUNX1) is also known as acute myeloid leukemia 1 protein (AML1) or core-binding factor subunit alpha-2 (CBFA2), a protein that in humans is encoded by the RUNX1 gene. Chromosomal translocations involving the RUNX1 gene are associated with several types of leukemia. Mutations in RUNX1 are implicated in cases of breast cancer. High expression of RUNX1 is associated with adverse survival of pancreatic cancer patients and has tumor promoting potential in pancreatic cancer.

Tumor specific markers are proteins which have been found to be expressed with qualitative or quantitative difference in tumor cells or tissues as compared to non-tumor cells or tissues. Qualitative difference means a difference in their sequence and/or structure as compared to the forms expressed in non-tumor cells; quantitative difference means in higher or lower amount as compared to the level of expression in non-tumor cells.

The inventor considered that most tumor markers are not 100% specific of tumor cells, which means that they are also expressed, under variable forms and amounts, in some non-tumor cells. This biological issue of lack of complete tumor markers specificity is a key cause of failure of anti-cancer treatments based on drugs.

The inventor considered that a strategy to overcome this obstacle should include:
Obtaining Early Cancer Detection through distinctive identification of cancer Giant Cells from non-cancer Giant Cells. Since noncancer Giant Cells are macrophage-like cells, Cancer Giant Cells can be identified a) if the Giant Cells express a tumor marker, such as telomerase, a syncytin 1 or 2, HERV proteins, CD71, racemase, RunX2, RunX1, PSMA or b) if the Giant Cells express a combination of markers not expressed by the non-Cancer Giant Cells, such as: epithelial and macrophage markers, epithelial and mesenchymal markers, PSA and macrophage markers, PSMA and macrophage markers. Epithelial markers are, for instance: AE1/AE3, KL-1, EpCAM, E-cadherin, macrophage markers are, for instance: CD11b, CD68, CD163, mesenchymal markers are, for instance, Vimentin.
Early detection of CTC/CGC (Tumor Cells/Cancer Giant Cells) thanks to their tumor markers allows early cancer prophylaxis or cancer treatment. Early prophylaxis or treatment will allow one to use lower doses/lighter schemes of treatment as the number or TC/CGC in the body is low, which will spare non-tumor cells. Killing CTC/CGC with low doses/light schemes of treatment is expected to eliminate them completely or decrease their number to a level that allows the body's immune system to get rid of them without eliminating non tumor cells.
Use of the CTC/CGC or the TC/CGC markers to apply/develop a TC/CGC-specific prophylaxis or treatment. TC/CGC-specific prophylaxis or treatment means the use of TC/CGC or TC's/CGC's tumor markers and/or combination of tumor markers and/or combination of tumor and non-tumor markers to eliminate or inactivate TC/CGC.

A method based on the detection of Cancer Giant Cells expressing telomerase or other cancer biomarkers can provide a sensitive and convenient way to detect cancer or cancer risk before the development of cancer symptoms identified by conventional diagnostic methods. Accordingly, the inventor focused efforts on isolation and identification of Giant Cells having diameters of at least 15 microns expressing telomerase and other cancer biomarkers, such as syncytin, Prostate-Specific Membrane Antigen (PSMA) and Racemase. The inventor studied whether detection of such Cancer Giant Cells could provide convenient and non-invasive means to detect early cancer risk and subsequent prophylactic or therapeutic treatment.

### BRIEF SUMMARY OF THE INVENTION

One aspect of the invention comprises a method for early detection of cancer or cancer risk by identifying or isolating Cancer Giant Cells having an axial diameter of 15 microns or greater and which express a cancer phenotype or genotype, such as expression of abnormally high amounts of telomerase, expression of syncytins 1 or 2, of syncytins receptors, of HERV proteins, of their receptors, or of various cancer antigens.

A related aspect involves the isolation of Giant Cells from a liquid biological sample by filtration, such as by Isolation by SizE of Tumor cells (ISET). Giant Cells having cancer genotypes or phenotypes may also be isolated by affinity-based methods using antibodies that bind to telomerase's peptides, syncytins, or cancer antigens on Cancer Giant Cells.

Another aspect of the invention is a method for treating a subject in which Cancer Giant Cells have been detected. The subject may be previously undiagnosed with cancer, at risk of being diagnosed or developing cancer, in remission or recovering from a prior cancer treatment, or previously diagnosed with a cancer. Treatment may target Cancer Giant Cells for elimination or inactivation, and/or other cancer cells. Treatments may include, but are not limited to radiation, surgical resection, or administration of an anticancer biologic or drug. A biological or drug treatment may comprise oligonucleotides, mRNA, miRNAs, or siRNAs, which target Cancer Giant Cells or comprise nucleic acid or protein conjugates that specifically eliminate or inactivate Cancer Giant Cells or other cancer cells in a patient in which Cancer Giant Cells have been detected.

Another aspect of the invention is a method for treating a subject at risk of developing cancer or already diagnosed with cancer and from whom Cancer Giant Cells have been detected by administering agents, such as antibodies, antibodies, including monoclonal antibodies, bi-specific and tri-specific antibodies, or other agents, like for instance CAR-T cells, that target antigens/receptors expressed by Cancer Giant Cells or targeted conjugates thereof, such as cytotoxic antibody conjugates (ADC: antibodies-drug conjugates) or nanotherapies, such as vectorized nanoparticles delivering drugs (Leng F. Strategies on Nanodiagnostics and Nanotherapies of the Three Common Cancers, NANOMATERIALS 8, 202, 2018).

The foregoing paragraphs have been provided by way of general introduction and are not intended to limit the scope of the following claims. The described embodiments, together with further advantages, will be best understood by reference to the following detailed description taken in conjunction with the accompanying drawings.

### BRIEF DESCRIPTION OF THE DRAWINGS

FIG. 1A (left): a circulating Cancer Giant Cell stained with MGG.

FIG. 1B (right). The same Cancer Giant Cell after immunostaining with telomerase. Blue arrows on top right indicate the Cancer Giant Cell (CGC). The red arrows on bottom left indicate a Circulating Tumor Microembolus (CTM), also called Tumor Cell Cluster.

FIG. 2. A multinucleated Cancer Giant Cells (major axis 60 microns) expressing syncytin.

FIGS. 3(a)-3(i) depict Giant cells from prostate cancer patients and healthy donors after Giemsa staining. FIGS. 3(a)-3(f) are representative images of the different PGCC morphologies observed in prostate cancer patients. Cell shapes: tadpole-shape in FIGS. 3(a), 3(e), 3(f); round shape in FIG. 3(b); globular shape in FIG. 3(c); amorphous shapes in FIG. 3(d). FIGS. 3(g)-3(i) are representative images of cells observed in healthy donors' urine samples.

FIGS. 4(a)-4(f): Representative images of normal cells present in urine samples. Cells stained with Giemsa's Azur eosin methylene blue solution. Top in FIGS. 4(a)-4(c) normal epithelial cells, bottom in FIGS. 4(d)-4(f) describe umbrella cells.

FIGS. 5(a)-5(e): Characterization of PGCC from prostate cancer patients by Giemsa staining and ICC. Left panels correspond to Giemsa staining, while middle panels show the same cells after ICC. FIG. 5(a) multinucleated PGCC expressing AMACR; FIG. 5(b) mononucleated PGCC in EMT, positive to epithelial (brown) and vimentin (pink) markers; FIG. 5(c) multinucleated PGCC strongly positive to TERT (nuclear and cytoplasmic positivity); FIG. 5(d) multinucleated PGCC strongly expressing PSMA (pink) marker; FIG. 5(e) mononucleated PGCC expressing the macrophage marker CD68 (brown) with strong PSMA expression (pink). Visualization system: DAB (brown) and permanent-red (pink). Color versions of these figures are available.

### DETAILED DESCRIPTION OF THE INVENTION

As disclosed herein this technology involves, inter alia, the isolation, identification, and/or characterization of Tumor Cells/Cancer Giant Cells (TC/CGC), such as Giant Cells (GC) expressing above normal levels of telomerase and/or various cancer antigens. As shown in the Examples below, the inventor's results showed that Cancer GC are characterized by telomerase hyperexpression with or without epithelial markers.

In order to identify Cancer GCs and distinguish them from non-cancer GCs, the inventor isolated and characterized GC in patients with different types of cancer as well as patients having inflammatory diseases. GCs were isolated and separated from other blood cells and blood components by filtration, thus isolating and enriching GCs having axial diameters of at least 15, preferably 40, microns from the patients. The inventor also identified on Giant Cells other cancer-associated markers including Telomerase, syncytins, CD71, and markers usually found on macrophages and/or epithelial cells, and MC1-R.

The discovery of telomerase hyperexpression as cancer marker of Giant Cells was unexpected because telomerase is hyper-expressed in actively proliferating cells, such as stem cells and non-giant tumor cells. Since Cancer Giant Cells are known to be barely proliferating cells, the hyperexpression of telomerase in Cancer Giant Cells, probably due to a cancer-associated gene expression dysregulation, was not sought and expected to be absent.

The discovery of syncytin as a cancer marker of Giant Cells was surprising because Syncytins are cell-cell fusion protein active in placental development. Syncytins genes are derived from endogenous retroviral elements and obtain novel functions through a process of convergent evolution.

Based on their findings that Cancer GCs express high levels of telomerase and macrophage markers, the inventor proposed to target Cancer GCs using Imetelstat, which is an oligo that interferes with telomerase function. Therapies which target tumor cells expressing telomerase are known, see Zanetti M A second chance for telomerase reverse transcriptase in anticancer immunotherapy NATURE REVIEWS|CLINICAL ONCOLOGY14: 115, 2017; Vonderheide, R. H., Telomerase as a universal tumor-associated antigen for cancer immunotherapy ONCOGENE, 2002, 21, 674-679 and Zhong-Li Liao, Antitumor effect of new multiple antigen peptide based on HLA-A0201-restricted CTL epitopes of human telomerase reverse transcriptase (hTERT) CANCER SCI 2012; 103:1920-1928 (incorporated by reference). The detection of CGC expressing increased levels of telomerase at early stages of cancer development or cancer recurrence would allow to treat patients with lower doses and/or shorter anti-telomerase treatments still able to eliminate them and stop cancer development. TERT vaccines, telomerase-specific antisense oligonucleotides, antibodies that bind telomerase peptides (ADC: antibodies-drug conjugates) are examples of anti-telomerase treatments. Anti-telomerase drugs can be targeted to telomerase expressing CGC by using multi-specific antibodies (Elshiaty M et al Principles and Current Clinical Landscape of Multispecific Antibodies against Cancer INT. J. MOL. SCI., 22, 5632, 2021) targeted to telomerase antigens and epithelial antigens or other antigens expressed by the CGC including macrophage-specific antigens, such as CD163, CD11b, or endothelial antigens, such as CD31 or mesenchymal antigens such as vimentin.

The inventor considered that Imetelstat or another telomerase blocking agent could be addressed specifically to Cancer GC, for instance, using bi-specific or tri-specific antibodies against two antigens of Cancer GC (such as, e.g., telomerase and epithelial antigens like EpCam) or telomerase and syncytin and/or its receptor, or telomerase and macrophage antigens or telomerase and cancer antigens like PSMA). Such targeted treatment of Cancer GCs could eradicate cancer by preventing cancer development or recurrence after an anticancer treatment or by eliminating cancer cells very early prior to conventional cancer diagnosis. In fact, Imetelstat induces bone-marrow suppression because bone-marrow stem cells and progenitors are proliferating cells and express telomerase. Some levels of telomerase activity are detected in tissues with high self-renewal capacity, such as the bone marrow, testes, gastrointestinal crypt epithelium, and hair follicles. Zanetti M A second chance for telomerase reverse transcriptase in anticancer immunotherapy NATURE REVIEWS|CLINICAL ONCOLOGY14: 115, 2017. Thus, there is a need to specifically target the Imetelstat or other anti-telomerase treatments to Cancer GC by using, for instance, bi-specific or tri-specific antibodies directed to two specific antigens of Cancer GC coupled with Imetelstat or other anti-telomerase treatments.

Another effective strategy involves using bi-specific, or tri-specific antibodies, called BiTEs (bifunctional T-cell engagers) with binding specificity for one or two CGC-associated antigens on one or two arms and, on the other arm, binding specificity for an activating receptor expressed by T cells (usually CD3). Longo D L. The Expanding Clinical Role of Bifunctional Antibodies. N. ENGL J MED 387; 24, 2022.

TC/CGC expressing telomerase can be targeted in a 100% specific manner by using, as combined targets, telomerase peptides and tumor antigens, such as syncytins, or telomerase peptides and non-tumor antigens which are not expressed by bone-marrow cells nor by other telomerase expressing cells (e.g., testes, gastrointestinal crypt epithelium, and hair follicles).

Using this strategy, the anti-telomerase treatment will eliminate TC/CGC expressing telomerase but will not eliminate bone marrow cells nor cells of testes, gastrointestinal crypt epithelium, and hair follicles which also express telomerase.

Other ways to use cancer TC/CGC as medicaments and/or develop anti-cancer GC therapy include: a) by generating Cancer GC-specific anti-idiotype vaccines, b) by targeting Cancer GC specifically with CAR-T cells, c) by targeting Cancer GC specifically with monoclonal antibodies, bi-specific or tri-specific antibodies, d) by using anti-viral therapies to Cancer GC expressing HERV proteins (such as syncytins) or their receptors, e) by using Cancer GC specific epigenetic drugs, by using vectorized nanoparticles targeted specifically to TC/CGC. Further methods and compositions which may be applied in connection with the diagnosis and treatment of cancers associated with Cancer Giant Cells includes those described by and incorporated by reference to:
Rurik J. et al. CAR T cells produced in vivo to treat cardiac injury. SCIENCE. 2022 Jan. 7; 375 (6576): 91-96.Tefferi A. et al. A Pilot Study of the Telomerase Inhibitor Imetelstat for Myelofibrosis. N ENGL J MED. 2015 Sep. 3; 373 (10): 908-19. Massumoto C. et al. Complete remission of mantle-cell non-Hodgkin lymphoma with a dendritic cell vaccine. HEMATOL ONCOL STEM CELL THER. 2009; 2 (1): 302-4.
Runcie K, Budman D, John V, Seetharamu N. Bi-specific and tri-specific antibodies—the next big thing in solid tumor therapeutics. MOL MED. 2018 Sep. 24; 24 (1): 50.
Pilla L, Ferroneb S, Maccallic C. Methods for improving the immunogenicity and efficacy of cancer vaccines. EXPERT OPIN BIOL THER. 2018 July; 18 (7): 765-784.
Schneider, D. et al. Trispecific CD19-CD20-CD22-targeting duoCAR-T cells eliminate antigen-heterogeneous B cell tumors in preclinical models. SCI TRANSL MED. 2021 Mar. 24; 13 (586): eabc6401.
Nobili A, Kobayashi A, Gedeon P, Novina C. Clutch Control: Changing the Speed and Direction of CAR-T Cell Therapy. J CANCER IMMUNOL (Wilmington). 2022; 4 (2): 52-59.
Filin I. et al. Recent Advances in Experimental Dendritic Cell Vaccines for Cancer. FRONT ONCOL. 2021 Sep. 23; 11:730824.
Chu D. et al. Recent Progress of Stem Cell Therapy in Cancer Treatment: Molecular Mechanisms and Potential Applications. CELLS. 2020 Feb. 28; 9 (3): 563.
Yu J, Sun H, Cao W, Song Y, Jiang Z. Research progress on dendritic cell vaccines in cancer immunotherapy. EXP HEMATOL ONCOL. 2022 Jan. 24; 11 (1): 3 and
Tapia-Galisteo A. et al. Trispecific T-cell engagers for dual tumor-targeting of colorectal cancer. ONCOIMMUNOLOGY. 2022 Feb. 7; 11 (1): 2034355.

Garrido, et al., Polyploid giant cancer cells are frequently found in the urine of prostate cancer patients, CANCERS, 2023, 15, 3366. As described by Garrido, et al cells of large size called PGCC (Polyploid Giant Cancer Cells) have emerged as a pillar in cancer development and progression, possibly being the “first cells” from which the cancer starts. PGCC have been studied in cancer tissues from patients and in laboratory models. They have also been found in the blood, occasionally. By applying a method able to detect rare cells in urine, we found these PGCC in the urine of patients with prostate cancer. No study has ever published this finding. Our work is preliminary but deserves to be shared with the scientific community as it opens the way for more studies targeting the role of these PGCC and their possible use as an early and non-invasive marker of prostate cancer development. Further details are incorporated by reference to Garrido, et al., supra.

All of the articles cited herein including those above are incorporated by reference.

The technology disclosed herein includes but is not limited to the following methods and procedures. A method for:
1) identification of Tumor Cells/Cancer GC in body fluids as a way for non-invasive, early cancer diagnosis and early cancer recurrence diagnosis.
2) use of Tumor Cells/Cancer GC to identify/define/develop a personalized, targeted therapy to eliminate them.
3) use of Tumor Cells/Cancer GC as companion diagnostic test to follow anti-cancer strategies and/or anti-cancer treatments.

For 1), GC are typically isolated from body liquids using a suitable method including one of the following methods: size-based isolation, density-based isolation, immune-mediated isolation, red blood cells lysis, white blood cells depletion/elimination. Tumor Cells/Cancer GC are identified using their large size (15 microns or more) combined with their expression of one or more tumor markers or tumor associated proteins including telomerase, syncytins, syncytins receptors, HERV proteins and their receptors, PSMA, Racemase (AMACR), CD 71 (transferrin receptor), AFP, tyrosinase, MIA S100, beta HCG, etc., see worldwideweb.antibodies-online.com/resources/18/1528/protein-tumor-markers. (incorporated by reference, last accessed Jan. 9, 2023). One or more of these markers may be used to isolate and identify Cancer Giant Cells.

For 2) a targeted immune-response to Tumor Cells/Cancer GC's molecules (cancer GC antigens, cancer GC epitopes, cancer GC neoantigens, cancer GC molecules) are obtained (a) by generating Tumor Cells/Cancer GC-specific mRNA for Tumor Cells/Cancer GC vaccination, (b) by generating Tumor Cells/Cancer GC-specific anti-idiotype vaccines, (c) by targeting Tumor Cells/Cancer GC specifically with CAR-T cells, (d) by targeting Tumor Cells/Cancer GC specifically with monoclonal antibodies, bi-specific or tri-specific antibodies, e) by using anti-viral therapies to Tumor Cells/Cancer GC expressing HERV proteins (such as syncytins 1 and 2), (f) by using Tumor Cells/Cancer GC specific epigenetic drugs, (g) by using nanotherapies, such as vectorized nanoparticles delivering drugs specifically targeting Tumor Cells/Cancer GC. One or more of these modes of treatment may be used to treat a subject from whom Cancer Giant Cells have been detected or isolated.

For 3) Tumor Cells/Cancer GC identification is used to follow patients after anti-cancer strategies or anti-cancer treatments and determine the need of surveillance or further anti-cancer strategies or anti-cancer treatments based on the presence or absence of Tumor Cells/Cancer GC. Ongoing surveillance may be used for subjects in whom Tumor Cells/Cancer Giant Cells have been detected.

Technical Terminology. Terminology used herein is for the purpose of describing particular embodiments only and is not intended to be limiting of the invention.

Tumor markers. A tumor marker, here, is a protein present in or produced by cancer cells. Tumor markers are generally not perfect. For instance, telomerase is expressed by the majority of cancers but also by bone marrow cells and other proliferating cells (see Zanetti M, 2017) (see Duffy M. Tumor markers in clinical practice: a review focusing on common solid cancers. MED PRINC PRACT. 2013; 22 (1): 4-1; Takamizawa S. et al. Diagnostic value of tumor markers in identifying favorable or unfavorable subsets in patients with cancer of unknown primary: a retrospective study. BMC CANCER. 2022 Apr. 14; 22 (1): 412. Both documents are incorporated by reference.

Non-tumor markers (also called cell markers). Cell markers refer to a type of marker that is specifically expressed at a specific time in a specific cell and can be used to identify specific cells and monitor cell growth and differentiation. Examples of cellular non-tumor markers include Mesenchymal Markers, such as Alpha-SMA, Fibronectin, N-Cadherin, S100A4, Slug, Snai, and Vimentin; Epithelial Markers such as Cytokeratins, Desmoglein-3, E-Cadherin, Laminin, MUC-1, Syndecan-1, and Beta-catenin; Macrophage markers including CD163, CD68, and CD11b.

Giant Cells (GC) also called multinucleated Giant Cells (MGC) or polyploid Giant Cells (PGC), are formed by endoreduplication or cell fusion and have been described in physiological and pathological conditions, including cancer onset and recurrence. In fact, cell polyploidy and cell fusion are physiological cellular processes essential for fertilization, viral entry, muscle differentiation and placental development, among others. The formation of GC can be triggered by long lasting stress conditions. Giant Cells thus include physiological and pathological or potentially pathogenic cells.

A Giant Cell may have an axial diameter ranging from 15, 20, 25, 30, 35, 40, 50, 60, 70, 80, 90, 100, 110, 120 or more microns (or other diameters disclosed herein). However, in some embodiments, Giant Cells of different sizes may be selected such as cells or multinucleated cells having diameters of at least 15 microns but less than 40 microns or refer to a cell produced by the union of two or more cells, such as by union of histiocytes forming granulomas or to cells having more than one nucleus.

Giant Cells (GC) are a heterogeneous cell type which has been described both in physiological and pathological conditions, including cancer. Cancer GC, also called Polyploid Giant Cancer Cells (PGCC), have been shown to be involved in cancer's onset and recurrence, therefore circulating PGCC could be used as sentinel cells for early cancer diagnosis and early cancer treatment. However, using circulating PGCC as sentinel cells heralding the presence or development of cancer or cancer recurrence implies finding a cancer marker of PGCC which demonstrates their tumor nature. In fact, circulating non-cancer Giant Cells can be detected in patients with chronic inflammation and in response to an infection, such as tuberculosis, herpes, HIV, or COVID or to a foreign body. There are no reports showing such universal cancer marker applied to circulating PGCC nor that circulating PGCC can be detected in a reliable, diagnostic and specific manner at a very early stage of cancer or cancer recurrence. Circulating Cancer Giant Cells are a subtype of Circulating Tumor Cells.

Cancer Giant Cells (CGC) are considered cells able to give origin to a cancer cell proliferation. They are thus considered to be potentially the first cancer cells. However, non-cancer Giant Cells also exist. The characteristics of all Giant Cells (cancer and non-cancer GC) include one or more of the following:
a) They are big cells.
b) They can have multiple nuclei or one big nucleus only.
c) They are polyploid.
d) They are generated by cellular stress.
e) They are considered non proliferating cells (or with a low proliferating activity).
f) They originate from endoreduplication or from cell fusion of two cells.
g) When they originate from fusion of two cells, the two cells can be of the same cell type or of two different cell types.
h) When cells of two cell types fuse, generally one cell is a monocyte/macrophage cell and the other is another cell type like an epithelial cell.

Non-cancer Giant Cells may be characterized as cells associated with cellular stress, chronic inflammation or infections. However, knowledge about the characteristics of cancer GC which allow to distinguish them from non-cancer GC is very poor.

Tumor Cells and Cancer GC (also called PGCC, Polyploid Giant Cancer Cells) are defined as cells capable of generating cancer.

Cancer, also including malignant cancer, is a potentially deadly, abnormal cell proliferation and differentiation. Cancer cells (also called Tumor Cells) are cells which can generate a cancer. They are distinguished from cancer-associated cells like CAML (Cancer Associated Macrophage like cells) or other cell types the presence of which is associated to cancer but which are not, themselves, cancer cells generating a cancer.

Giant Cells named CAML have extensively been reported in patients with cancer and in patent applications (Adams D and collaborators). However, the markers found to be expressed by CAML or other similar cells are not universal cancer markers, thus cannot be used in patients with very early-stage cancers, i.e., in seemingly healthy patients. According to the present disclosure, the proof that GC are Tumor Cells/Cancer GC (or PGCC) is given by their expression of the universal cancer marker telomerase or other cancer specific markers. Typically, Cancer Giant Cells are cells which have major axis dimension equal to or larger than 15 microns, and preferably equal or larger than 40 microns. They are considered to be cells capable of giving origin to a cancer or a recurrence of cancer after anti-cancer treatment; therefore, their identification in blood as cancer Giant Cells, through a cancer-specific marker such as Telomerase marker, allows to diagnose the presence of a cancer in a seemingly healthy subject or subject in cancer remission and to use the cancer-specific marker in circulating GC, along with other Giant Cells' markers, as a therapeutic target. The present invention also relates to treatment of subjects in which such circulating Tumor Cells/Cancer GC have been identified in order to eliminate them and stop cancer formation or recurrence.

Distinctive characteristics of Cancer GC as compared to non-cancer GC include one or more of the following:

a) CGC are poly-aneuploid.

b) When formed by fusion of two cells, at least one of them is a tumor cell, the other cell is generally a macrophage or an endothelial cell.

c) They can give origin to proliferating cancer cells by “budding”; they are then called “pregnant giant cells”.

Cancer types include both liquid and solid tumors, neoplasms, or cancers associated with the presence of Giant Cells. Cancer types include solid cancers such as breast, prostate, pancreatic, NSCLC, sarcoma, kidney, bladder, colon, colorectal, uterine sarcoma, neuroblastoma, esophageal, ovarian, melanoma, liver, head & neck, glioblastoma, and lung cancer and liquid cancers such as leukemia, lymphoma, and myelodysplastic Syndrome.

The methods disclosed herein advantageously provide early diagnosis of cancers associated with, or which are characterized by presence of Tumor Cells/Cancer Giant Cells or where such CGCs act as cancer cell reservoirs.

Very early cancer or cancer recurrence detection is defined as diagnosis or detection of preclinical cancer, i.e. detection of cancer in an individual without any sign or symptoms of cancer or cancer recurrence, i.e. detection of cancer in a seemingly cancer-free individual.

Detection at very early stage thus means cancer or cancer recurrence detection before the cancer mass is diagnosed using the classical diagnostic approaches; e.g., imaging such as MRI, CT-scan, ultrasound, Rx, biopsy, or surgery using conventional methods known as of the effective filing date of this application. At this so early stage, Circulating Tumor Cells (CTC)/Cancer GC have to be distinguished from non-cancer GC in order to be used for cancer diagnostic/detection and therapeutic purposes. In some embodiments, very early cancer detection is made 0.25, 0.5, 1, 2, 3, 4, 5, 6, 7, 8, 9, 10, 11, 12, 18, 24 or more months before a patient is diagnosed with cancer or with relapsing or recurrent cancer using conventional methods or before the diagnosis or onset of locally advanced cancer, which has grown outside the tissue or body part where it started, or metastatic cancer. Very early detection of cancer also includes detection of cancer before stage 1, 2, 3 or 4 cancer, advanced stage cancer, late-stage cancer, secondary cancer or recurrent cancer. For example, very early detection of cancer may take place at stage TO where there is no evidence of a tumor or at stage TX which there is no information about a tumor or where the presence of a tumor cannot be measured.

Biological samples include, but are not limited to, liquid and solid samples which contain cells. Preferably, a liquid sample is a biological liquid that is easy to obtain such as blood or buffy coat cells isolated from blood is used. Giant Cells and Cancer Giant Cells can be isolated from tissue, blood and other fluids of a subject, where the cells contain molecular changes associated with primary/secondary tumors, or molecular changes that are independent of the tumors but remain a clinical target of interest. Typically, these mutations are used in determining early detection, prognostic, diagnostic or predictive information of cancer. As formation of tumor cells, such as Cancer Giant Cells, is directly associated with tumor growth, progression and spread, the ability to detect molecular changes found within the cell may directly correlate with treatment. The cells (or their naked nuclei) can be purified from blood in most solid malignancies, in non-solid tumors, and in premalignant conditions so that the cells and naked nuclei can then undergo molecular characterization. In the procedures disclosed herein whole Giant Cells or their nucleic acids, which may be separately isolated, may be used to assess some types of genotypic or phenotypic features.

Liquid biopsy is a laboratory test done on a sample of blood, urine, plasma, serum or other body fluid such as saliva, sputum, cerebrospinal fluid (CSF) and seminal plasma to look for cancer cells from a tumor or small pieces of DNA, RNA, or other molecules released by tumor cells into a person's body fluids. Liquid biopsy allows multiple samples to be taken over time, which may help doctors understand what kind of genetic or molecular changes are taking place in a tumor. A liquid biopsy may be used to help find cancer at an early stage. It may also be used to help plan treatment or to find out how well treatment is working or if cancer has come back. Current liquid biopsy methods for cancer detection include collecting and analyzing circulating cancer cells and/or extracellular vesicles/exosomes/circulating nucleic acids obtained from cancer cells or cancer-associated cells, such as cells isolated from a subject's blood. These cells/extracellular vesicles/exosomes and/or nucleic acids are subjected to molecular analysis, such as determination of mutations and/or epigenetic/methylation modifications to cancer cell nucleic acids, with the resulting data used to screen for cancer, and to diagnose and treat cancer; see US 2020/0049713 A1 (incorporated by reference). Any of such liquid biopsy methods may be used in the methods disclosed herein.

Expression and hyperexpression. A Cancer Giant Cell may express higher amounts of a particular protein such as telomerase or a macrophage antigen compared to Giant Cells in subjects not at risk of, or not having cancer. For example, a Cancer Giant Cell may express 1, 2, 5, 10, 20, 30, 40, 50, 60, 70, 80, 90 or 100 wt. % (or mole %) or more telomerase or macrophage antigens compared to a normal subject not at risk of cancer or to a normal subject with an inflammatory condition or infection. Alternatively, some Cancer Giant Cells may express lower amounts of antigens than cells or Giant Cells from subjects not at risk of cancer. Circulating Cancer Giant Cells may be obtained from biological liquids such as blood in which the Giant Cells circulate.

ISET. The ISET® technology (ISET: Isolation by SizE of Tumor cells) is a highly innovative patented technique implemented with the Rarecells Device and Consumables, which allows the lossless isolation from blood of Circulating Rare Cells, including Circulating Tumor Cells, while preserving their integrity, thereby allowing their analysis by different methods, including Blood Cytopathology. The ISET® technology is based on a simple blood draw with no harm or pain to patients.

Cancer-associated biomarkers. Cancer GC inter alia are characterized by macrophage or epithelial markers and telomerase hyperexpression. Other markers found by the inventor include syncytins and MC1-R. Macrophage antigens which are expressed by tumor cells include CD163, MAC387, and DAP 12; see Shabo, I, et al., Expression of macrophage antigens by tumor cells, Adv Exp Med Biol., 2011, 714, 141-150 incorporated by reference.

Telomerase also called terminal transferase, is a ribonucleoprotein that adds a species-dependent telomer repeat sequence to the 3′ end of telomeres; hypertext transfer protocol secure://en.wilikepedia.org/wiki/Telomere. The molecular composition of the human telomerase complex was determined by Scott Cohen and his team at the Children's Medical Research Institute (Sydney Australia) and consists of two molecules each of human telomerase reverse transcriptase (TERT), telomerase RNA (TR or TERC), and dyskerin (DKC1). The genes of telomerase subunits, which include TERT, TERC, DKC1 and TEP1, are located on different chromosomes. The human TERT gene (hTERT) is translated into a protein of 1132 amino acids. TERT polypeptide folds with (and carries) TERC, a non-coding RNA (451 nucleotides long). TERT has a ‘mitten’ structure that allows it to wrap around the chromosome to add single-stranded telomere repeats.

Chromosomes are capped by the nucleoprotein structures called telomeres, which are involved in several important functions such as the regulation of gene expression, recombination, mitosis and meiosis. Telomeres protect chromosome termini from eliciting the DNA damage response and play a fundamental role in longevity and cell proliferation. Most telomeres carry 3′ overhangs that can be generated by incomplete synthesis at the lagging strand after degradation of the RNA primer of the distal-most Okazaki fragment and resection, and/or by resection of a blunt-end telomere intermediate resulting from leading strand synthesis. This end-replication problem is solved by an enzyme called telomerase. While a deficit in telomerase would translate into shorter telomeres over each replication, triggering senescence, its continued or dysregulated expression could unbalance growth control, potentially leading to oncogenesis, if control mechanisms do not direct the cell e.g., to apoptosis. Therefore, telomerase abnormal expression is considered a universal cancer marker. Telomerase expression detection has been applied to circulating tumor cells. However, it has never been used to distinguish circulating Giant Cells, PGCC from circulating non-cancer GC in the aim to perform a very early detection of cancer or cancer recurrence.

Antibodies or nucleic acid probes, which bind to telomerase proteins and nucleic acids may be used to quantify its expression or quantity.

Drugs and therapies that target telomerase. include Imetelstat, an oligo that interferes with telomerase function. Other agents including miRNA, siRNA or other oligonucleotides that target expression of telomerase may be used. Other drugs and therapies include those described by Guterres, A. N., et al., Targeting telomerase for cancer therapy, ONCOGENE, 2020, 39, 811-5824 (2020) which is incorporated by reference. Approaches to targeting telomerase include:

Immunotherapies-Peptide or DNA vaccines supply immunogenic TERT epitopes that stimulate immune responses against telomerase-expressing cancer cells. Adoptive cell transfer therapies entail the infusion of telomerase-specific cytotoxic T-cells.

Direct telomerase inhibitors-small molecules can bind to TERT and inhibit its catalytic activity resulting in gradual telomere attrition. Alternatively, oligonucleotides complementary to the TERC template region can act as competitive telomerase inhibitors.

G-quadruplex (G4) stabilizers disrupt telomerase function by blocking the resolution of telomeric G-quadruplex DNA.

Incorporation of nucleoside analogues into newly synthesized telomeres impairs POT1 binding, causing telomere dysfunction that elicits a DNA damage response and cell death.

Targeting TERT gene expression-TERT promoter mutations (TPMs) generate novel binding sites for ETS transcription factors that reactivate TERT expression in cancer. Targeting regulation of the mutant TERT promoter represents an emerging approach.

Disrupting telomerase localization-interference with telomerase recruitment mediated by TCAB1 and shelterin subunits elicits telomere dysfunction.

Immunotherapy successfully treats some kinds of cancer, such as melanoma. This treatment involves manipulating a human's immune system to destroy cancerous cells. Humans have two major antigen identifying lymphocytes: CD8+ cytotoxic T-lymphocytes (CTL) and CD4+ helper T-lymphocytes that can destroy cells. Antigen receptors on CTL can bind to a 9-10 amino acid chain that is presented by the major histocompatibility complex (MHC). HTERT is a potential target antigen. Immunotargeting should result in relatively few side effects since hTERT expression is associated only with telomerase and is not essential in almost all somatic cells. GV1001 uses this pathway. Experimental drug and vaccine therapies targeting active telomerase have been tested in mouse models, and clinical trials have begun. One drug, Imetelstat, is being clinically researched as a means of interfering with telomerase in cancer cells. Most of the harmful cancer-related effects of telomerase are dependent on an intact RNA template. Cancer stem cells that use an alternative method of telomere maintenance are still killed when telomerase's RNA template is blocked or damaged.

Two telomerase vaccines have been developed: GRNVAC1 and GV1001. GRNVAC1 isolates dendritic cells and the RNA that codes for the telomerase protein and puts them back into the patient to make cytotoxic T cells that kill the telomerase-active cells. GV1001 is a peptide from the active site of hTERT and is recognized by the immune system that reacts by killing the telomerase-active cells. Such a vaccine may be administered to subjects in which Cancer GCs have been identified but who do not yet manifest other symptoms of cancer.

Another independent approach is to use oligoadenylated anti-telomerase antisense oligonucleotides and ribozymes to target telomerase RNA, reducing dissociation and apoptosis. The fast induction of apoptosis through antisense binding may be a good alternative to the slower telomere shortening.

siRNAs are small RNA molecules that induce the sequence-specific degradation of other RNAs. siRNA treatment can function similar to traditional gene therapy by destroying the mRNA products of particular genes, and therefore preventing the expression of those genes. A 2012 study found that targeting TERC with an siRNA reduced telomerase activity by more than 50% and resulted in decreased viability of immortal cancer cells. Treatment with both the siRNA and radiation caused a greater reduction in tumor size in mice than treatment with radiation alone, suggesting that targeting telomerase could be a way to increase the efficacy of radiation in treating radiation-resistant tumors.

Drugs and therapies that target macrophage antigens or other biomarkers expressed by Cancer Giant Cells. Macrophage antigens which are expressed by tumor cells include CD163, CD 68, CD 11b, MAC387, and DAP 12; see Shabo, I, et al., Expression of macrophage antigens by tumor cells, Adv Exp Med Biol., 2011, 714, 141-150 incorporated by references. Drugs and agents which target these antigens or biomarkers include antibodies to cancer associated antigens expressed by Cancer Giant Cells including cytotoxic antibody conjugates.

Telomerase length. Telomere length can impact the efficacy of telomerase-directed therapy. Time taken for telomerase inhibitors to exert anticancer effects is expected to depend on initial telomere length since the length of the shortest telomere in a cell dictates the onset of telomere dysfunction. In some embodiments of the invention telomerase length in isolated Giant Cells having cancer-associated phenotypes or genotypes may be measured and correlated with treatment, regression or progression of a cancer associated with Cancer GCs.

The term isolated means separated from components in which a material is ordinarily associated with, for example, an isolated Giant Cell can be separated from some or substantially all red blood cells, plasma, and other components of a blood or other biological sample; or it may be separated from 5, 10, 20, 30, 40, 50, 60, 70 80, 90, 95, <100, or 100% of other non-Giant Cells in a sample, or from 5, 10, 20, 30, 40, 50, 60, 70 80, 90, 95, <100, or 100% of Giant Cells not over-expressing telomerase or from Giant Cells not expressing one or more of the macrophage, epithelial, or other cancer associated antigens or proteins disclosed herein.

### Example 1

### Isolation and Characterization of New Biological Markers for Cancer Giant Cells.

The work described in the following examples shares the immunocytochemical methodology described below and refers to data in Table 1.

Immunocytochemistry. ISET membrane spots were cut out and used for single or dual-color immunocytochemistry for CTC and PGCC identification and characterization according to the manufacturer's instructions. The spots on ISET membranes were submitted to Giemsa staining and digitalized using an Olympus scanner BX-61VS. Subsequently the Giemsa stain was washed for 5 min with PBS1X on a soft shaker. Antigen retrieval was then performed for 20 min at 50° C. using En Vision™ FLEX Target Retrieval Solution High pH (50×) (Dako Omnis, Agilent). After brief washing with PBSIX, permeabilization was performed for 5 min in Triton 0.1% PBSIX solution. The immunocytochemistry was performed using EnVision™ G|2 Double stain System, Rabbit/Mouse (DAB+/Permanent Red) (Agilent technologies). The spots were incubated, in ISET® ICC Staining Box, one hour at room temperature with specific antibodies diluted with DAKO Antibody Diluent (Agilent technologies). After immunostaining, cells were counterstained with hematoxylin for 3 min and the spots were scanned using an Olympus scanner BX-61VS. Negative and positive controls were performed for each ICC staining (Table 1). For negative controls, cells from cell lines negative to the tested antigen were spiked in healthy donors' blood, filtered, and used as follows: by omitting the primary antibody, to ensure the exclusion of cross-reactivity; and by including the primary antibody, to guarantee the specificity of the antibody. For positive control, cell lines positive for the tested antigen were spiked in healthy donors' blood, filtered, and immunostained. Table 1 shows the antibodies list used in this study and the cell lines used. Giant Cells were defined as cells having the largest diameter of 40 microns or more. They were identified with Giemsa staining and counted. After immunocytochemistry, cells were individually analyzed by visualizing side by side the cytomorphological (Giemsa staining) and the immunostaining details.

TABLE 1

| Antibodies | Source | Positive control | Negative Control |
|---|---|---|---|
| Macrophages panel | | Primary macrophages | A549 cell line |
| Recombinant Anti-CD163 antibody | abcam | direved from ThP1 | |
| [EPR1 [symbol] 51 [symbol] ] | | | |
| Recombinant Anti-CD68 antibody | abcam | | |
| [EPR20545] | | | |
| Recombinant Anti-CD11b antibody | abcam | | |
| [EP1348Y] - C-terminal (ab52478) | | | |
| Epithelial panel | | A549 cell line | Blood cells from |
| Ep-CaM (C-10) | Santa Cruz | | healthy donor |
| E-Cadherin(G-10) | Santa Cruz | | |
| Cytokeratin KL-1 | Diago [symbol] i [symbol] s | | |
| Monoclonal mouse cytokeratin | DAKO | | |
| AE1/AE3 | | | |
| CD61 (integrin beta 3) Recombinant | Thermofisher | MEG-01 cell line | A549 cell line |
| Rabbit Monoclonal Antibody ( [symbol] 19-09) | scientific | | |
| CD41 mouse Monoclonal Antibody | Thermofisher | MEG-01 cell line | A549 cell line |
| (CRC64) | scientific | | |
| Anticorps mouse monoclonal | Santa Cruz | Bone Morow slide | Blood cells from |
| RUNX2 (F-2) | | section from AML patient | healthy donor |
| Anti-Telomerase reverse | abcam | Hela cell line | Blood cells from |
| transcriptase antibody (ab230527) | | | healthy donor |
| Anti-Syncytin 1 polyclonal | Cliniscience | Human normal Placenta | Blood cells from |
| antibody | | | healthy donor |

[symbol]

indicates data missing or illegible when filed

### Example 2

### Sentinel Circulating Polyploid Giant Cancer Cells Overexpressing Telomerase Allow Early Diagnosis of Cancer in a Seemingly Healthy Subject.

Despite decades of research work and billions of dollars spent to fight cancer, cancer morbidity and mortality remains high. Cancer patients continue to die as early detection and targeted treatments of patients at risk of developing cancer or who are in early stages of cancer are limited and not sufficiently effective. A new vision of cancer detection and treatment and new strategies and weapons to treat and eliminate cancer are needed. In view of the above, the inventor focused efforts on the identification and characterization of Giant Cells in subjects at risk of having cancer, not yet diagnosed with cancer, or having cancer.

Giant Cells (GC) have heterogenous characteristics and have been associated with various physiological and pathological conditions, including cancer. Cancer GCs, also called Polyploid Giant Cancer Cells (PGCCs), have been shown to be involved in the onset and recurrence of cancer. However, there are no reports showing the detection of circulating PGCC at a very early stage, before the detection of the cancer mass. With an objective of developing a new method for early detection of cancer, the inventor sought to determine whether GCs could be detected in otherwise healthy subjects who were subsequently diagnosed with cancer. As disclosed herein, the inventor identified a seemingly healthy woman in which circulating PGCC were detected 5 years before the diagnosis of a Stage 1 cervical cancer. PGCC were detected repeatedly in the woman's blood along with circulating tumor cells (CTC) and sometimes with circulating tumor microemboli (CTM). However, all circulating cancer cells disappeared following the surgical ablation of the cancer.

The detection and characterization of PGCCs and CTCs allowed the inventor to define some of their main morphological and immunotypic characteristics. As determined by the inventor, PGCCs can circulate in blood at the very beginning of the cancer formation, thus confirming in vivo, in a patient, their very early appearance in the process of cancer development. The inventor also characterized circulating PGCCs, thus providing a novel framework and guide for sensitive early detection of cancer and development of early targeted cancer treatments, such as treatments that eliminate PGCCs and stop development and spread of cancer. Tumors may be described and modeled as an ecosystem which comprises normal somatic cells, cancer cells, and other tissues and components of the tumor microenvironment (Pienta K J. Cancer cells employ an evolutionarily conserved polyploidization program to resist therapy. SEMINARS IN CANCER BIOLOGY 81:145-159, 2022). GCs may be keystone components of this ecosystem. By analogy, in many ecosystems, the community structure and ecosystem integrity are dependent on a single and often low-abundant species termed the keystone species from the architectural keystone of an arch. If the keystone is removed, the ecosystem arch collapses. Keystone species thus exert a disproportional large effect on the ecosystem relative to their abundance. Cancer GCs may act as keystone species and/or as reservoirs of cancer cells.

The cancer ecosystem could be dependent upon a keystone species: a rare population of cells that has the capacity to survive the harsh conditions of the tumor microenvironment (e.g., hypoxia, low nutrients, low pH), to metastasize, and to mediate therapeutic resistance by surviving treatment and then repopulating tumors with resistant cancer cells. While these keystone cancer cells survive, metastatic cancer will remain incurable. Accordingly, the inventor sought to identify and eliminate the keystone cancer cells, thus permitting the tumor ecosystem to collapse by making the bulk of tumor cells vulnerable to traditional therapies and opening the door for a cancer cure. There are now convergent data strongly suggesting that the role of keystone species is played, in the cancer ecosystem, by Polyploid Giant Cancer Cells. As disclosed herein, the inventor shows for the first time that PGCC can be present in the body and circulate in blood before a tumor mass becomes detectable. The results presented here confirm that PGCC appear in the early steps of cancer development and could therefore play the role of keystone species of the cancer ecosystem. They increase our knowledge and fuel our interest in PGCC detection and targeted elimination.

### Patient, Materials and Methods.

Patient. A 63-year-old woman, aware of the scientific publications issued using ISET, asked in 2012 to be followed by periodic analyses of her blood using ISET. She did not have any familiarity of cancer, nor exposure to carcinogens, no other known risk for cancer nor for viral infection (HIV, HPV, HBV, HCV) associated with cancer. She did not have any risk of cancer except age.

Blood samples (10 mL) were collected repeatedly from a seemingly healthy woman, born in 1954, starting from 2012 to 2022 upon her written informed consent. No pathological symptoms nor abnormal pathological exams were reported during the follow up, in particular no bleeding, no pain, no fatigue, no anemia, and no imaging exams abnormalities.

The ISET pilot test results showed no abnormal cells of any type from 2012 to 2015 in 8 different ISET tests. Starting from 2015, a PGCC and some CTM appeared in blood. See Table 2A. In the following years, the number of CTC and of PGCC was variable with period increases. CTM also appeared and increased in number at certain dates.

TABLE 2A
Blood samples were collected in EDTA
tubes at the times shown in Table 2A

| Date | Number of GC | Number of CTC |
|---|---|---|
| Oct. 2nd, 2015 | 1 | 0 |
| Jun. 8th, 2016 | 3 | 0 |
| Nov. 6th, 2017 | 20 | 45 |
| Dec. 13th, 2017 | 5 | 0 |
| Apr. 19th, 2018 | 4 | 0 |
| Nov. 13th, 2018 | 2 | 1 |
| Dec. 12th, 2019 | 23 | 46 |
| Jan. 15th, 2020 | 2 | 3 |
| Feb. 5th, 2020 | 5 | 6 |
| Jun. 2nd, 2020 | 27 | 45 |
| Jun. 18th, 2020 | 8 | 3 |
| Jul. 1st, 2020 | 1 | 1 |
| Oct. 23rd, 2020 | 1 | 1 |
| November 2020 | 1 | 0 |
| Dec. 23rd, 2020 | 4 | 1 |
| Feb. 1st, 2021, A | 1 | 1 |
| Feb. 1st, 2021, B | 11 | 18 |
| Feb. 4th, 2021 | 3 | 1 |
| Mar. 16th, 2021 | 1 | 0 |
| April 2021 | 2 | 0 |

Table 2A. Detection by ISET of circulating Giant Cells and CTC in 10 ml of blood and corresponding date. HPV51 was found at the cervix in December 2020. A colposcopy was performed in January 2021 followed by conization on February 1^{st} Blood samples were obtained on February 1^{st} before (A) and after conization (B). The number of CGC after conization represents an iatrogenic spreading and not a spontaneous CGC circulation. Hysterectomy was performed on Mar. 18, 2021. After April 2021 no CGC nor CTC were found in ISET tests performed every 3 months.

After blood collection, tubes were stored at room temperature for subsequent analysis by Isolation by SizE of Tumor cells (ISET) (Rarecells Inc US) according to manufacturer instructions. ISET methodology isolates intact GC, CTCs and CTM from blood through direct filtration without using antibodies, thus exploiting the larger size of GC, CTC and CTM as compared with leukocytes and uses polycarbonate membrane with 8-μm-diameter cylindrical pores. The ISET platform is EC marked and has technical and clinical validation (Laget S, et al. Technical Insights into Highly Sensitive Isolation and Molecular Characterization of Fixed and Live Circulating Tumor Cells for Early Detection of Tumor Invasion. PLOS ONE. 2017; 12 (1): e0169427). The inventor used procedures of this platform and CTC definition criteria according to previous detailed reports. Véronique J. Hofman, Cytopathologic Detection of Circulating Tumor Cells Using the Isolation by Size of Epithelial Tumor Cell Method AM J CLIN PATHOL 2011; 135:146-156.

Circulating tumor microemboli (CTM) were defined as groups or clusters of tumor cells containing three or more distinct nuclei and previously identified in metastatic cancer patients. Two types of PGCCs were described: multinucleated and mononucleated giant cancer cells; Mirzayans R, et al., Multinucleated Giant Cancer Cells Produced in Response to Ionizing Radiation Retain Viability and Replicate Their Genome. INT J MOL SCI. 2017; 18 (2) . . . . The common morphological characteristics of PGCCs are large nuclei (or multinuclei), and their average size is more than three times larger of diploid cancer cells; Ohashi R, et al. Breast carcinoma with osteoclast-like Giant Cells: A cytological-pathological correlation with a literature review. ANN DIAGN PATHOL. 2018; 33:1-5. The nuclei of PGCCs are usually irregular, and the morphology of PGCCs varies. In this work, the inventor considered PGCC cells with the biggest diameter equal or larger than 40 microns.

Since the pilot ISET test cannot identify the organ from which the tumor cells derive, extensive screenings and imaging analyses were performed (see Table 2B).

TABLE 2B

| Year | Screening analyses | Result |
|---|---|---|
| 2019 | Pap test, IRM breast, IRM | All negative |
| | pelvis, colonoscopy | |
| 2020 | Pet-scan, cf-DNA, Rx chest, | All negative |
| | Pap test | |
| 2020 December | Pap test HPV | positive |
| 2021 January | Colposcopy | positive |

All gave a negative result for the search of a tumor mass. In December 2020, her GP decided to prescribe a second PAP test, despite the previous one, performed in May 2020, was negative. The second Pap test was done collecting cells more deeply in the cervical canal, thus beyond the transition zone. The Pap test was coupled with the search of the HPV genome. The results of the Pap test showed the presence of atypical cells and the HPV genome analysis showed the presence of HPV 51. A colposcopy was performed in January 2021. The biopsy analysis showed the presence of a neoplastic proliferation. A conization was performed on February 1^{st} and the histopathological analysis showed a cervical cancer stage IA.

Based on these results, the patient underwent a hysterectomy on Mar. 18, 2021. In the following blood controls, every three months, no circulating tumor cells or PGCC were found in her blood. HPV genomes were detected by smear analysis 3 months after hysterectomy but disappeared at the following control 6 months after surgery. Given the results of the smear analyses and blood analyses the patient was considered cured only by surgery, without need of any chemotherapy or radiation therapy.

Prior to cancer diagnosis of a seemingly healthy patient with imaging, Giant Cells were isolated from the patient's blood. Patient blood was determined to contain tumor cells and Giant Cells. These Giant Cells were designated as Cancer Giant Cells on the ground that the same patient did not have Giant Cells before the identification of Giant Cells and Circulating Tumor Cells (CTCs) in her blood; and because the cellular and nuclear characteristics of these Giant Cells were abnormal and tumor-like manifesting anisokaryosis, anisocytosis, irregular nuclear outlines, and coarse chromatin, hyperchromasia, enlarged and hyperchromic nucleic high N/C ratio, hyperchromatic nucleic and thickened cell membrane further indicating a cancer origin.

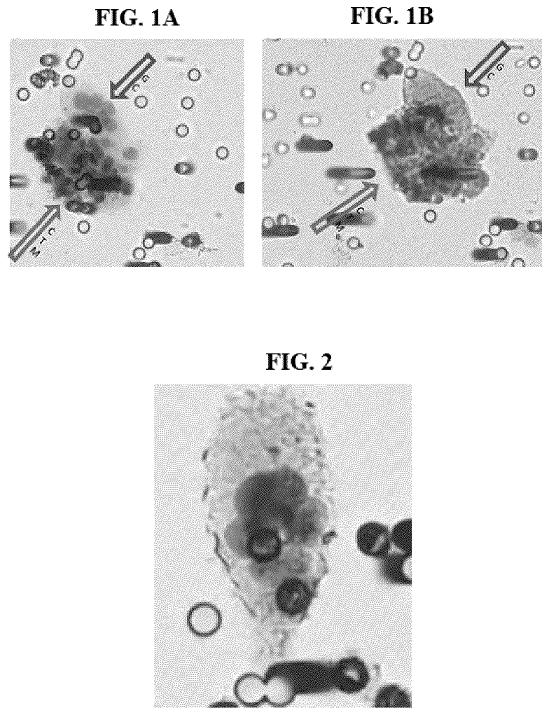

Furthermore, these Giant Cells were positive for the telomerase biomarker as shown in FIGS. 1A and 1B. Some GC also expressed macrophage and epithelial markers (data not shown). The patient was diagnosed with cervical cancer. Polyploid giant cancer cells (PGCC) were visualized by MGG and characterized by the conglomeration of overlapping nucleic showing anisokaryosis, condensed chromatin and poorly visible nucleoli. FIG. 1B shows the same PGCC after staining with an antibody specific for telomerase and was shown to strongly express telomerase. The small black circles depict pores of an ISET membrane. See FIGS. 1A and 1B.

FIGS. 1A and 1B. Left (FIG. 1A): a Giant Cell stained with MGG. Right (FIG. 1B): the same Giant Cell after immunostaining with telomerase. Blue arrows at upper right indicate the Giant Cell (GC). The red arrows at lower left indicate a Circulating Tumor Microembolus (CTM), also positive for telomerase.

PGCC immunolabelling (FIGS. A and 1B). The ICC analyses allowed to demonstrate that circulating PGCC are strongly positive to the Telomerase marker, to the cocktail epithelial markers and some of them are positive to the cocktail macrophage marker and weakly or strongly positive to the vimentin marker.

Analysis. The results obtained by the inventor show that Cancer Giant Cells, also called PGCC, Polyploid Giant Cancer Cells, are found in blood at the very beginning of cancer development, 5 years before cancer was detected, in stage IA, through conventional approaches. Polyploid giant cancer cells (PGCCs) are cells with multiple nuclei or a single giant nucleus containing multiple sets of chromosomes. The mechanism leading to formation of PGCC may be a consequence of endoreplication, which is related to genetic or physical disturbances of mitosis (including: endocycling, mitotic slippage, endomitosis or cytokinesis failure), cell fusion or cell cannibalism; Pienta K J, et al. Cancer recurrence and lethality are enabled by enhanced survival and reversible cell cycle arrest of polyaneuploid cells. PROC NATL ACAD SCI USA. 2021; 118 (7). Pienta K J, et al. Poly-aneuploid cancer cells promote evolvability, generating lethal cancer. EVOL APPL. 2020; 13 (7): 1626-34.

On the other hand, polyploidy, the multiplication of the whole genomes, has been shown to act as either a barrier or a driver of tissue repair and regeneration in the development and diseases. Polyploid Giant Cells that arise via endoreduplication and/or cell fusion have been well described. Various physiologic states or pathologic conditions, such as hypoxia, starvation, temperature, and aging, induce cells to exit their mitotic cell cycle and differentiate into polyploid cells.

PGCC have been demonstrated to be somatic equivalent of blastomeres. This means that they can have embryonic-like stemness and can generate “new type of cells”, which are, in fact, cancer cells. As a matter of fact, we found that circulating PGCC associated to the earliest steps of cancer development strongly express Telomerase. To our knowledge, telomerase hyperexpression has never been described in PGCC.

Human telomerase reverse transcriptase (hTERT), the catalytic subunit of telomerase, is highly expressed in over 85% of cancer cells to maintain the immortality of the cancer cells but is not active in most normal somatic cells. Therefore, hTERT can be an ideal candidate not only to distinguish PGCC from non-cancer polyploid cells, but also as druggable tumor associated antigen (TAA) in PGCC. In fact, unlike most other TAA, the expression of hTERT in tumor cells is associated with tumor growth, development and invasion. Targeting hTERT may also minimize immune-escape of PGCC because the inhibition of telomerase activity in hTERT-positive tumor cells leads to telomere shortening and should induce PGCC death by apoptosis.

As disclosed herein, the expression of telomerase in PGCC was detected at very early steps of cancer formation in humans and paves the way to a targeted, very early treatment and elimination of PGCC now considered to be at the origin of cancer.

### Example 3

Determination that Syncytin is a Marker for Cancer Giant Cells

The inventor used ISET to isolate CTC and GC from blood, identified CTC and Cancer Giant Cells in the blood of a patient with breast cancer and found that they were positive for syncytin. FIG. 2 describes circulating multinucleated Giant Cells (major axis 60 microns) expressing syncytin.

Syncytin is a fusogenic protein which is physiologically expressed by trophoblastic cells making them fuse into syncytiotrophoblastic cells and plays a key role in placenta formation. Syncytin expression has already been described in certain cancers, but it has not previously been described as being expressed by, and being a marker for, circulating cancer Giant Cells nor in Circulating Tumor Cells or CTM.

Syncytin is one of the proteins which can be expressed by reactivated HERV (Human Endogenous retroviruses) which make 8% of our genome. Since syncytin is fusogenic and GC can derive from cellular fusion the inventor investigated whether it was expressed in Cancer GC and have now demonstrated its presence in Cancer GCs. Further description of syncytin and other tumor antigens are described by Gasent Blesa J, Candel V. E CANCER MEDICAL SCIENCE. 2009; 3:145; Grandi N, Tramontano E. HERV Envelope Proteins: Physiological Role and Pathogenic Potential in Cancer and Autoimmunity. FRONT MICROBIOL. 2018 Mar. 14; 9:462. Larsson L, Bjerregaard B, Wulf-Andersen L, Talts J. SCIENTIFIC WORLD JOURNAL. 2007 Aug. 17; 7:1193-7; Liu C. et al. Upregulation of syncytin-1 promotes invasion and metastasis by activating epithelial-mesenchymal transition-related pathway in endometrial carcinoma. ONCOTARGETS THER. 2018 Dec. 17; 12:31-40; Zhou Y. et al. Implication of human endogenous retrovirus W family envelope in hepatocellular carcinoma promotes MEK/ERK-mediated metastatic invasiveness and doxorubicin resistance CELL DEATH DISCOV. 2021 Jul. 8; 7 (1): 177; Alcazer V. et al. HERVs characterize normal and leukemia stem cells and represent a source of shared epitopes for cancer immunotherapy. AM J HEMATOL. 2022 September; 97 (9): 1200-1214; Díaz-Carballo D. et al. Cytotoxic stress induces transfer of mitochondria-associated human endogenous retroviral RNA and proteins between cancer cells. ONCOTARGET. 2017 Oct. 7; 8 (56): 95945-95964 (each incorporated by reference).

The inventor also considered and proposed that Cancer Giant Cells expressing syncytin 1 or syncytin 2 or HERV proteins can be treated with anti-viral therapies, including non-toxic antiviral drugs, to eliminate Cancer Giant Cells and avoid or treat nascent cancers; see Diaz-Carballo et al. Therapeutic potential of antiviral drugs targeting chemorefractory colorectal adenocarcinoma cells overexpressing endogenous retroviral elements. Journal of Experimental & Clinical Cancer Research (2015) 34:81; Mikkel Dons Müller et al. A Systematic Review of Expression and Immunogenicity of Human Endogenous Retroviral Proteins in Cancer and Discussion of Therapeutic Approaches Int. J. Mol. Sci. 2022, 23, 1330.

### Example 4

### Polyploid Giant Cancer Cells are Frequently Found in the Urine of Prostate Cancer Patients

Patients and Methods. Urine from healthy patients and patients with prostate cancer were evaluated. The inventor noticed that large multinucleated and mononucleated cells were frequently found in patients with prostate cancer.

The inventor thus comparatively assessed at a single point in time the presence and features of GC detected in single urine samples from patients with clinically significant prostate cancer and from healthy subjects. Samples were studied from 45 patients with clinically significant prostate cancer followed at the Urology and Oncology departments of Cochin Hospital, Paris, France, and at the Urology department of Foch Hospital, Suresnes, France. Informed consent was obtained from all subjects before they provided a urine sample (Institutional Review Board-approved protocol, code 2020-A02712-37). No clinical follow-up was planned. All patients had been diagnosed with prostate adenocarcinoma by biopsy before or well, after urine collection (see below). The majority of patients had a Gleason score of 7 or higher ( 44/45=97.8%) and 13 out of 45 (28.9%) patients had metastasis. Metastases were diagnosed by imaging (bone scan). The average PSA serum level was 39.0 ng/mL. See Table 3 for clinical characteristics.

TABLE 3
Clinical and pathological characteristics
of 45 patients with prostate cancer.

| Clinical Parameter | Number (%) or Median (Range) |
|---|---|
| Age * average (range) | 73 (55-94) |

Serum PSA (ng/mL)

| | |
|---|---|
| Average | 39.0 |
| Median | 12.0 |
| Range | 3.34-275 |
| Unknown | 2 patients |

Gleason score

| | |
|---|---|
| 6 | 1 (2.2%) |
| 7 | 18 (40.0%) |
| ≥8 | 26 (57.8%) |
| Metastasis | 13 (28.9%) |

* years.

The inclusion criteria for prostate cancer patients were patients with newly diagnosed and untreated prostate cancer, not having been diagnosed with any other type of tumor before the inclusion; patients with metastatic prostate cancer on any imaging; and patients with high serum PSA level and/or a high risk of prostate cancer having French Social Security affiliation and agreeing to participate in this work. Forty-three healthy subjects without known prostatic pathology such as benign prostatic hyperplasia (BPH) or prostatitis, under 50 y.o., and agreeing to participate in this work were also included. Healthy subjects had an average age of 32.4 y.o., ranging from 20 to 49. Informed consent was obtained from all subjects.

Urine Sample Collection and Stabilization. Single voided urine samples were obtained from patients with clinically significant prostate cancer prior to any treatment and without any short-term previous prostate stimulation such as digital rectal examination, prostatic massage, or prostate biopsy. There were 20 samples collected before prostate biopsy, while 25 samples were collected 2-4 weeks after biopsy. No urine samples presented macroscopic hematuria. Voided urine samples, including the first urine catch, were collected at any time of the day, directly into a suitable container, and fixed with modified Saccomanno's fixative within the first 30 min after collection. Fixed urine samples were stored for up to 7 days. No predetermined urine volume was required for this exploratory study. The samples' volume varied from 10 mL to 200 mL. After fixation, urine samples were conserved at room temperature and treated in the following 24 h or conserved at 4° C. for up to 7 days before treatment.

Human Cell Lines. PC-3 (prostate; PSA positive), VCaP (prostate; PSMA and PSA positive), LNCaP (prostate; PSMA, AMACR, and TERT positive), DU-145 (prostate; EpCAM positive), A375 (melanoma; vimentin and epithelial cocktail positive), HeLa (cervix; TERT and weak CD163 positive), and Daudi (B lymphoblast; negative control for all tested markers) cell lines were selected as positive and negative controls for the different ICC protocols (optimization and routine) (Table 4). The cell lines were purchased from ATCC® (Manassas, VA, USA). Those cell lines were cultured in a complete medium containing DMEM with 10% FBS and 10 U/mL penicillin and streptomycin (Life Technologies, Waltham, MA, USA) in a 5% CO₂ atmosphere at 37° _C. Cells were passaged every 3-5 days according to the growing speed. After cell detachment with trypsin, the cells were counted using Trypan Blue, added to urine samples collected from healthy volunteers, then fixed with modified Saccomanno's fixative for at least 30 min and finally treated by ISET® (Rarecells Diagnostics, Paris, France); or added to DPBS and fixed with 1.8% formaldehyde for 2 min at room temperature and processed alone.

TABLE 4
Primary antibodies used for cell characterization.

| | | Control | | |
|---|---|---|---|---|
| | | Positive | Staining | |
| Antibody | Type | Cell Line | Pattern | Supplier |
| PSMA | Mouse | VCaP, | Membranous/ | Agilent (Dako, Santa Clara, |
| ( [symbol] E6) | monoclonal | LNCaP | cytoplasmic | United States), M3620 |
| AMACR | Rabbit | LNCaP | Cytoplasmic | Agilent (Dako, Santa Clara, |
| (13 [symbol] ) | monoclonal | | granular | United States), M3616 |
| Panel- | Rabbit | HeLa | Cytoplasmic | Abcam (Cambridge, UK), |
| Macrophage | monoclonal | | | ab254013 |
| markers | | | | |
| (CD11b, CD68, | | | | |
| CD163) | | | | |
| V [symbol] ( [symbol] P20) | Rabbit | A375 | Cytoplasmic | ThermoFisher Scientific |
| | monoclonal | | | (Waltham, MA, USA), |
| | | | | MA5-14564 |
| Panel-Epithelial | Mouse | A375, | Cytoplasmic and | Aligent, M [symbol] 51 [symbol] |
| markers | monoclonal | DU-145 | membranous | Santa Cruz Biotechnology |
| (AE1/AE3, KL-1, | | | | (Dallas, TX, USA), |
| EpCAM, | | | | [symbol] C-58825, SC-2530 [symbol] , |
| E-cadherin) | | | | [symbol] C-8425 |
| TERT (2D8) | Mouse | LNCaP, | Nuclear/ | Thermo Fisher, |
| | monoclonal | HeLa | cytoplasmic | MA [symbol] -16033 |
| PSA (EP1588Y) | Rabbit | PC-3, | Cytoplasmic/ | Invirogen, (Waltham, MA, |
| | monoclonal | VCaP | membranous | USA), MA [symbol] - [symbol] 4471) |

[symbol]

indicates data missing or illegible when filed

Urine Processing. Fixed urine samples (volumes ranging from 10 mL to 200 mL) were processed through a proprietary method using a suitable ISET® device and disposable cartridges (Rarecells Diagnostics, Paris, France). Briefly, urine was directly loaded into the cartridge without previous centrifugation nor sedimentation and treated at −10 kPa. After processing, ISET® membranes were dried and stored at −200° C. until use.

Cells Analysis—Giemsa Stain and Immunocytochemistry (ICC). A specific protocol was applied in order to analyze cell morphology and ICC labeling on the same cell. ISET® membranes containing fixed cells from urine or human cell lines spiked in urine from healthy subjects were stained with Giemsa's Azur eosin methylene blue solution (Merck 1.09204, Darmstadt, Germany), then digitized using the VS200 Slide Scanner (Olympus-Life Science, Hamburg, Germany). Digital analysis (including cell count and cell size measurements) was performed using Olympus Image Analysis software version 2.1 (Hamburg, Germany). Subsequently, cells were immunolabeled by ICC and then ISET® membranes were digitized again. Comparative side-by-side image analyses of the same cells with cytological staining and with immunolabeling were carried out using Olympus Image Analysis software. ICC protocols after Giemsa staining were applied to better-characterized GC. To this aim, ISET® membranes were submitted to dual color ICC using EnVision™G|2 Double stain System, Rabbit/Mouse (DAB+/Permanent Red, Agilent, Dako, Santa Clara, CA, USA).

Briefly, cells were hydrated with DPBS for 1 min. Double and single ICC protocols were started with 10 min of heat-induced antigen retrieval at 70° C. Cell permeabilization was performed with Triton 0.1% in DPBS. Endogenous peroxidases were inactivated for 10 min. Primary antibodies (Table 4) were incubated for 1 or 2 h according to each protocol. After that, the polymer/HRP reagent was incubated for 15-30 min. DAB+Working Solution. Briefly, cells were hydrated with DPBS for 1 min. Double and single ICC protocols were started with 10 min of heat-induced antigen retrieval at 70_C. Cell permeabilization was performed with Triton 0.1% in DPBS. Endogenous peroxidases were inactivated for 10 min. Primary antibodies (Table 2A) were incubated for 1 or 2 h according to each protocol. After that, the polymer/HRP reagent was incubated for 15-30 min. DAB+Working Solution was added for detection. After three washes with distilled water, spots were dried at room temperature. For the double-stain protocol, ISET® membranes were immediately washed with washing buffer solution, the double-stain block solution was added and incubated for 10 min, second antibodies were incubated for 1 or 2 h, the Rabbit/Mouse (LINK) solution for 10 min, followed by 30 min with the polymer/AP reagent. Finally, the permanent redworking solution was applied for signal detection. Side-by-side images of the same cell after Giemsa staining and ICC labeling were submitted to cytopathological analysis. Aside from the GC, we did not count all the cells on the spots, nor did we normalize the analysis based on urine cellularity or urine volume.

### Morphological Characterization of Polyploid Giant Cancer Cells in Urine Samples.

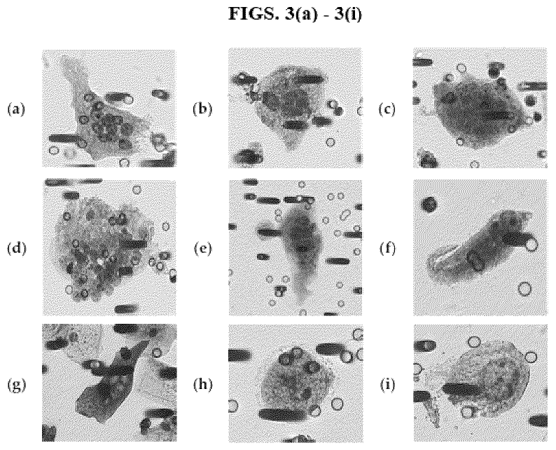

To investigate the presences of PGCC in urine samples, the cytomorphological characteristics of urinary cells enriched from 45 men diagnosed with prostate cancer and 43 healthy men 49 y.o. or younger were analyzed. PGCC were defined as cells with size equal or larger than 50 μm (maximum axis) and with cancer-like morphological characteristics: mononucleated or multinucleated cells (>10 nuclei) with nuclear hyperchromatism, high nucleo-cytoplasmic ratio, atypical nucleoli (number and size), and atypical cell shape and/or anisonucleosis. Morphological analysis of urine samples from patients with prostate cancer revealed variable GC phenotypes including globular, oblong, spindle-like, round, tadpole, and amorphous shapes, FIGS. 3(a)-3(f). In healthy men, GC phenotypes included amorphous, round, globular, and oblong shapes (FIG. 1g-i). Multinucleated cells with 10 or more nuclei were observed exclusively in patients with prostate cancer (6 out of 25 patients, 24%) and were considered to be GC with malignant features. GC from healthy subjects showed the presence of a regular and thickened outer contour of cytoplasm and similarities to urothelial cells. See FIG. 3 (a) which depicts Giant cells from prostate cancer patients and healthy donors after Giemsa staining. (a-f) are representative images of the different PGCC morphologies observed in prostate cancer patients. Cell shapes: (FIGS. 3(a), 3(e), 3(f) tadpole; 3(b) round shape; 3(c) globular; 3(d) amorphous 3(g0-3(i). are representative images of cells observed in healthy donors' urine samples.

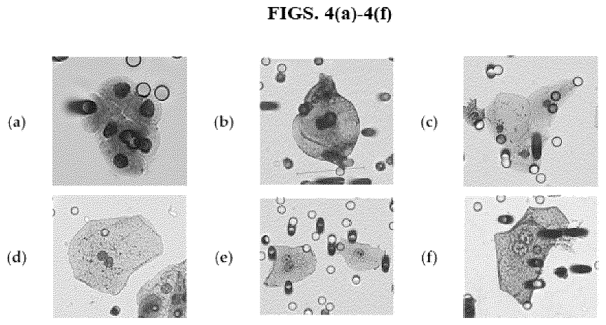

Despite their frequency and size of 50 microns or larger (FIG. 4(a)-4(f)), we did not count normal squamous epithelial cells (mainly from the distal urethra), urothelial cells (transitional epithelium from renal pelvis, ureters, bladder, and proximal part of the urethra), or cells with cytomorphological characteristics of umbrella cells (superficial layer covering basal and intermediate urothelial cells in the bladder) as GCs. Transitional cells from the intermediate and basal layer are characterized by round nuclei, smooth nuclear membrane and high N/C ratio due to the small amount of cytoplasm (FIG. 4(a)-4(c) Squamous epithelial cells are the largest cells in the urine sediment and are often considered as “contaminants” in urine cytology. They are flat cells with a single small, condensed nucleus, an opaque and uniform cytoplasm, and well-defined borders (FIG. 4(c)). Umbrella cells are derived from the most superficial cell layer in the bladder urothelium. They are characterized by a finely vacuolated, transparent, textured cytoplasm (compared to squamous cells) and double or multiple nuclei with at least one prominent small nucleolus, FIGS. 4(d)-4(f). Umbrella cells can be multinucleated but, in general, with no more than five nuclei. Highly multinucleated cells may be observed in instrumented urinary specimens (e.g., bladder washing or brushing.

A correlation between the quantity and/or type/characteristics of urinary cells and urine volume was not observed. The morphological analysis allowed the inventor to identify 64 GC with malignant features thus, PGCC in 25 out of 45 patients (55.6%) with prostate cancer. In those patients, the inventor also observed 64 GC with uncertain malignant features. In addition, it was observed, in a further 7 patients, 47 GC exclusively with uncertain malignant features (without PGCC by morphology). On the other hand, it was observed 23 GC with suspected malignant features (10 GC from 6 subjects) and with uncertain malignant features (13 GC from 5 subjects) in a total of 11 out of 43 healthy men (see Table 5).

TABLE 5
Table showing the total number of cancer patients and healthy
subjects with urinary giant cells and with PGCC identified
by cytomorphology and ICC (N° of cells in parentheses).

| | | With Urinary | |
|---|---|---|---|
| | Number | Giant Cells | With Urinary PGCC |
| Prostate cancer patients | 45 | 32 (175) | 22 (50) |
| Healthy donor | 43 | 11 (23) | 1 (1) |

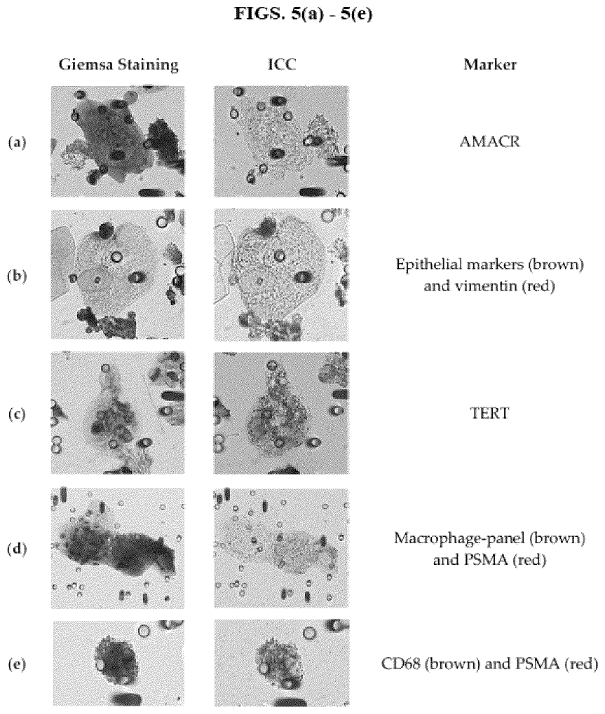

Immunocytological Characterization of Polyploid Giant Cancer Cells in Urine Samples. As reported for the methods described above, an individual cell, side-by-side analysis of cytological and immunocytological aspects (FIG. 5(a)-5(e)) was performed to help the identification of PGCC. In fact, immunostaining often masks the cytomorphological aspects which can be the key to identifying tumor-like features. The inventor observed that GC with malignant features often express macrophage-panel markers (CD68, CD163, CD11b) or epithelial markers (AE1/AE3, KL-1, EpCAM, E-cadherin) in combination with a second marker. PGCC were found to express cancer-related markers such as α-methylacyl-CoA racemase (AMACR) (FIG. 5(a)), or telomerase reverse transcriptase, TERT, FIG. 5(c). Other PGCC were found in epithelial-to-mesenchymal transition (EMT), as they expressed both epithelial markers (+) and vimentin (+) (FIG. 5(b). Finally, some multinucleated PGCC were positive to prostate-specific membrane antigen (PSMA), thus confirming their tumor nature and prostatic origin (FIG. 5(d)). We observed PGCC co-expressing macrophage (CD68 or macrophage-panel) markers and PSMA (FIG. 5(e), possibly derived by the fusion of a macrophage cell with a prostate tumor cell.

The cytopathological criteria required to identify tumor cells are known to be strict. Therefore, when they are not clear-cut, or are only partially present, the cells are defined as having uncertain malignant features. In gynecological cytology, cells with uncertain malignant features are defined as ASCUS (Atypical Squamous Cell of Unknown Significance) because it is difficult to classify them as tumor or non-tumor cells. However, a large study has demonstrated that, for ASCUS cells' correct classification, the combination of cell morphology with immunolabeling (double labeling p16-Ki67) is more accurate than either cell morphology or immunolabeling alone. In the setting of cells with uncertain malignant features, the use of complementary techniques such as ICC can help the cells' classification. The inventor applied the same approach of combination of cytopathological criteria and immunolabeling characterization to confirm or not the tumor feature observed by cytopathology and to classify GC with uncertain malignant features. By using the combination of tumor cytomorphology and ICC showing the presence of tumor-like markers, either EMT markers (epithelial+/vim+), PSMA, AMACR, or TERT, we found 50 PGCC from 22 prostate cancer patients, with an average number of 2.3 (range from 1 to 10) (see Table 5. Besides PGCC, it was also observed, in prostate cancers patients, the presence of smaller cells (<50 μm) expressing PSMA, AMACR, or other tumor markers, in variable amounts. Interestingly, we found the presence of large (<50 μm) macrophage-like cells in the urine of prostate cancer patients. The proportion of prostate cancer patients with PGCC was similar when urine was collected before (45%, 9 out of 20) versus after (52%, 13 out of 25) biopsy, showing that the PGCC we found after biopsy were not spread by the procedure. In fact, the urine sample was collected 2-4 weeks after biopsy. The ICC analysis of GC from heathy subjects using EMT markers, PSMA, and AMACR scored negative in all cases except in 1 healthy subject, who was found with 32 AMACR+GC cells, thus classified as PGCC. It was not known if these PGCC are derived from a prostate cancer since they did not express prostate markers (PSA or PSMA) In two healthy subjects, the inventor also found 5 GC with EMT markers but normal cytomorphological features. Furthermore, no cell in those samples had malignant or uncertain malignant features. Thus, we did not consider the 5 GC as PGCC because EMT can also be a marker of cell renewal.

Example 4 shows that giant cells (GC) having the characteristics of Polyploid Giant Cancer Cells (PGCC) are found in the urine of patients with prostate cancer. PGCC are rare cells that have been identified mostly in tumor tissues and studied by in vitro analyses. Some GC (called CAMLs) have also been found in the blood. Despite their big size, it is not trivial to distinguish them, in human samples, from non-cancer GC, mainly macrophages or inflammatory GC. However, extensive studies in cancer patients and in vitro have identified some GC as cancer cells able to give rise to tumors and tumor recurrence. The inventor and her team works in the field of liquid biopsy, in particular, in the isolation and characterization of circulating tumor cells and circulating tumor GC from blood. In this setting, this work was extended to the research of prostate cancer-derived tumor cells in the urine of patients with prostate cancer. As a first observation, it was found that the presence of GC more frequently in the urine of prostate cancer patients than in control subjects. We thus conducted a detailed immune-morphological study of these GC to identify or exclude their tumor nature. Giant polynucleated cells are very rare in urine; they can be superficial urothelial cells, including umbrella cells, macrophages, or cancer GC. Umbrella cells (also known as facet cells or superficial cells) are highly differentiated bi- or multinucleated cells, with a size ranging from 25 μm to 250 μm in diameter located in the bladder urothelium.

The total number of epithelial cells in urine (squamous and transitional cells) varies among men. Giant multinucleated umbrella cells (with up to 10 nuclei) can be seen in urine but this finding is definitely rare. By the way, atypical umbrella cells can be found in the urine of patients with urothelial cancer and be of diagnostic help; however, they have never been described in the urine of patients with prostate cancer and, in any case, umbrella-like cells in urine have never been associated with prostate cancer. We defined as PGCC those cells with size equal or larger than 50 μm and having cancer-like cytomorphological aspects (such as anisonucleosis, nuclear hyperchromatism, high nucleo-cytoplasmic ratio, atypical nucleoli, and atypical cell shape), also expressing tumor markers (TERT, PSMA, AMACR, vimentin and epithelial markers, etc.) detected by immunolabeling, the side-by-side cytomorphological and immunolabeling study helped us to identify the distinctive features of PGCC.

During this work by the inventor, some GC with uncertain malignant features according to morphological analyses, were shown to express tumor markers such as TERT, AMACR, or PSMA. As an example, the cell shown in FIG. 5(a) could be mistakenly considered an atypical umbrella cell, but its expression of the AMACR marker showed its highly probable malignant nature.

We have previously demonstrated the high specificity and limited sensitivity of cytomorphology in the field of circulating tumor cells. By studying the single cell morphological and genetic characteristics of CTC from patients with VHL-positive clear cell renal carcinoma, we found that cytomorphology had 100% specificity but much lower sensitivity. In that study, we observed that all the cells with malignant features carried the VHL mutation in single cells' analyses, thus were genetically proven tumor cells. However, also a relevant proportion of cells with uncertain malignant features were found to carry the same VHL mutation that was found blindly in the tumor tissue, showing that the presence of incomplete malignant features does not exclude the tumor cell nature. The same seems possibly true for some PGCC that we detected in urine. The EMT phenotype, which consists in the expression of epithelial markers and vimentin (epithelial+/vim+), is considered a tumor hallmark. However, it can also be related to embryonic development, skin wound healing, tissue regeneration, and renal fibrosis For example, EMT is required for re-establishing the skin barrier after a wound. Moreover, it has been shown in the context of bladder cell renewal after surgery. In healthy subjects, the inventor found 5 GC with EMT markers but normal cytomorphological features. No cell in those samples had malignant or uncertain malignant features. Thus, these cells were not considered as PGCC, but as cells with features associated with cell renewal. We found one healthy subject with 32 AMACR+GC cells, which we thus classified as PGCC.

Interestingly, we found some PGCC similar to the already-described PGCC generating daughter tumor cells by budding, see FIG. 3 (d). This characteristic of PGCC has been reported in vitro in primary cultures of ovarian cancer and in cell lines derived from leukemic cells. It is thought to be involved in the origin of PGCC-derived proliferating tumor cells in patients with cancer recurrence Although it is difficult to definitively demonstrate PGCC budding by images of cells detected in biological liquids from patients, published studies clearly indicate this possibility. To our knowledge, this is the first report showing PGCC “budding-like” images in urine samples.

As shown above, Example 4 provide clinical evidence that prostate cancers release PGCC into the urine. PGCC can be detected non-invasively and thus advantageously can be used to detect prostate cancer through a simple urine analysis. This work shows for the first time that PGCC with macrophage and PSMA markers are present in the urine of patients with prostate cancer. As explained, this work aimed to investigate if cancer giant cells, also called PGCC, are present in the urine of patients with prostate cancer (Gleason score equal or higher than 7). With direct PGCC counting this work found the presence of PGCC in 22 out of 45 (50%) cancer patients and 1 out of 43 healthy subjects, showing that cancer giant cells are present and detectable in the urine of prostate cancer patients.

### EMBODIMENTS

A method of early cancer detection and treatment or personalized or targeted treatment comprising, consisting essentially of, or consisting of (a) screening a subject for cancer, comprising detecting circulating Cancer Giant Cells or Giant Cancer Cells in a liquid or other biological sample from a subject previously determined to be at risk for cancer, or at risk for cancer recurrence, wherein said detecting comprises, consists essentially of, or consist of (aa) isolating Giant Cells that have a major axis diameter of at least 15 microns from a biological sample of a subject and (bb) detecting at least one cancer cell phenotype or genotype in the isolated Giant Cells from step a), thereby identifying Cancer Giant Cells or circulating Cancer Giant Cells; and (b) optionally treating the subject with a therapy to eliminate the cancer detected using the Cancer Giant Cells or circulating Cancer Giant Cells having a cancer cell phenotype or genotype identified in step (bb). In some embodiments, the method detects Giant Cells, Giant Cancer Cells, and/or Circulating Cancer Giant Cells.

In some embodiments, the biological sample is treated to remove red blood cells; or the Giant Cells are isolated from the biological sample by size exclusion or filtration; or the Giant Cells are isolated by passing the biological sample through a filter having a pore size that retains the Giant Cells.

In some embodiments of this method of early cancer detection, the subject has not been previously diagnosed with cancer and in other embodiments the subject has been diagnosed with cancer, is in remission from cancer, or is being monitored for progression or regression of cancer.

In some embodiments of this method of early cancer detection, the biological sample comprises a liquid such as a bodily fluid such as blood, urine, sperm, prostatic fluid or seminal fluid or a diluted or processed liquid sample containing the Giant Cells or Cancer Giant Cells or circulating Cancer Giant Cells. In other embodiments, it may comprise plasma, serum, or buffy coat cells. The biological sample may comprise macrophages, monocytes, lymphocytes, neutrophils, basophils, eosinophils, platelets, or other white blood cells. In another embodiment, the liquid or biological sample comprises urine or another non-blood biological fluid.

In other embodiments of this method of early cancer detection the cancer cell phenotype or genotype comprises cells that express at least one macrophage antigen, at least one epithelial antigen, telomerase or its subunits (e.g., TERT or TERC), a syncytin, a HERV protein, CD71, AFP, Racemase, PSMA, MC1-R, CD 163, MAC 87 or DAP12. In some embodiments, the cancer cell phenotype or genotype comprises aneuploidy, abnormal copy number variation (CNV), mutations, or epigenetic modifications compared to a non-cancerous control cell. The cancer cell phenotype or genotype may comprise an increased level of mRNA encoding telomerase or a subunit of telomerase compared to a non-cancerous control cell; or an increased level of telomerase.

In other embodiments, the at least one cancer cell phenotype comprises an increased level telomerase or a subunit of telomerase compared to a non-cancerous control cell; or an increased level of hTERT compared to a non-cancerous control cell; or increased expression of telomerase or its subunits. The treating in step (b) may comprise administering a drug, biologic, agent, or immunotherapy that targets telomerase or its subunits.

In another embodiment of the method of early cancer treatment the at least one cancer cell phenotype or genotype comprises an increased level of mRNA encoding a syncytin compared to a non-cancerous control cell; or comprises an increased level a syncytin compared to a non-cancerous control cell; or comprises an increased level syncytin 1 or syncytin 2 compared to a non-cancerous control cell. In some embodiments, the at least one cancer phenotype comprises increased expression of a syncytin and wherein the treating in step (b) comprises administering a drug, biologic, agent, antiviral, or immunotherapy that targets syncytin.

In another embodiment of the methods disclosed herein, the subject has not been previously diagnosed with cancer and the cancer detected using the Cancer Giant Cells biomarker identified in step (bb) allows cancer treatment in a timely manner.

In another embodiment of the methods disclosed herein, the subject has not been previously diagnosed with cancer recurrence and the cancer recurrence detected using the Cancer Giant Cells cancer cell-associated phenotype or genotype identified in step (bb) allows a timely personalized cancer treatment.

In another embodiment of the methods disclosed herein, the subject is being monitored for progression or regression of cancer and the Cancer Giant Cells or circulating Cancer Giant Cells cancer cell-phenotype or genotype identified in step bb) allows to establish cancer progression and need of timely cancer treatment.

In some embodiments of the methods disclosed herein, circulating tumor cells (CTCs) may further be detected.

Advantageously, the methods disclosed herein provide a personalized, targeted, timely treatment of cancer or cancer cells specific to the cancer cells in a particular subject. Such a treatment can be tailored to specific genetic and molecular characteristics of the Giant Cancer Cells detected by the methods disclosed herein. Once cancer cell markers characteristic of Cancer Giant Cells or CTCs are detected, a specific treatment for that type of cancer characterized by such markers can be immediately or timely used to treat, reduce the load, reduce the severity, or eliminate the Cancer Giant Cells or CTCs. Such a personalized, targeted treatment may provide higher efficacy than current one-size-fits-all approaches. Since the treatment targets a patient's cells bearing specific or unique cancer cell markers to that patient it may provide faster eradication or superior control of cancer. The methods disclosed herein provide easy and early ways to surveil and treat cancer potentially leading to long-term remission or cure of the cancer.

In another embodiment, the method of early cancer treatment is directed to detection and treatment of prostate cancer. In this method the biological sample is often urine the at least one cancer cell phenotype comprises an increased level of telomerase or its subunit(s), PSMA, syncytin, and/or racemase compared to a non-cancerous control cell. In some embodiments, the at least one cancer phenotype comprises increased expression of a telomerase and the treating in step (b) comprises administering a drug, biologic, agent, antiviral, or immunotherapy that targets telomerase or its subunits. In another embodiment, the at least one cancer phenotype comprises increased expression of a syncytin and wherein the treating in step (b) comprises administering a drug, biologic, agent, antiviral, or immunotherapy that targets syncytin.

Another aspect of the invention is drawn to a kit for detecting Circulating Cancer Giant Cells or Giant Cancer Cells, to be used for early cancer and early cancer recurrence detection, management of cancer treatment, determination of therapy and of treatment response is disclosed, comprising at least one antibody or other ligand that binds to a cancer marker on a Cancer Giant cell, or at least one probe or primer that can bind to or amplify a biomarker on a Cancer Giant Cell or circulating Giant Cancer Cell and optionally containers for said antibodies or ligands, for said probes or primers, reagents for detection of said biomarkers, instructions for use, or other components, supplies or equipment for detecting said biomarkers.

Another aspect of the invention is directed to a method of early cancer detection comprising screening a subject for cancer, comprising detecting Giant Cancer Cells or circulating Cancer Giant Cells in a biological sample from a subject previously determined to be at risk for cancer, or at risk for cancer recurrence, wherein said detecting comprises: (aa) isolating Giant Cells that have a major axis diameter of at least 15, 20, 25, 30, 35, or 40 microns from a biological sample of a subject; (bb) detecting at least one cancer cell phenotype in the isolated Giant Cells from step (aa), thereby identifying Cancer Giant Cells or circulating Cancer Giant Cells. Some embodiments of this method further comprise treating the subject a type of cancer expressing the cancer cell phenotype identified by step (bb).

Other embodiments are described and listed below.

One aspect of the technologies described herein is directed to a method of assaying for cancer-associated genotypes or phenotypes in Giant Cells said method comprising, consisting essentially of, or consisting of, obtaining Giant Cells from a biological sample of a subject, and analyzing the Giant Cells for cancer-associated phenotypes or genotypes, thereby identifying Giant Cells having a cancer-associated genotype or phenotype, wherein said Giant Cells have a diameter of 15, 20, 25, 30, 35, 40, 45, 50, 55, 60, 65, 70, 75, 80, 85, 90, 95, 100, 110, 120, 130, 140, 150, 160,170, 180, 190, 200, 300, 400, 500, 750, to 1,000 microns or more. Giant cells can result by a process of cell fusion and sometimes can range in size up to 1,000 microns or more. Advantageously, the Giant Cells will have diameters or average diameters of 40 microns or more.

In some embodiments of this method, the cancer-specific phenotype or genotype comprises expression by a Giant Cell of a tumor marker such as telomerase, PSMA, alpha fetoprotein (AFP), syncytins, racemase, CD71, etc. and/or at least one cancer-associated genotype and/or cancer cell phenotype.

In some embodiments of this method, the cancer-associated phenotype or genotype comprises expression by a Giant Cell of at least one macrophage antigen and/or at least one epithelial antigen and/or a cancer-associated marker like syncytin 1 or 2, syncytins receptors, CD71, HERV proteins and their receptors, MC1-R, CD 163, MAC 87 and/or DAP12.

In other embodiments, the cancer-associated phenotype or genotype comprises expression by the Giant Cell of at least one mRNA encoding a macrophage antigen, encoding an epithelial antigen, encoding syncytin 1 or 2, or syncytin receptors, or encoding HERV proteins and/or their receptors, CD71, racemase, PSMA, MC1-R, CD163, MAC 87 or DAP12.

In another embodiment, the cancer-associated phenotype or genotype comprises increased expression of telomerase or a subunit thereof compared to a Giant Cell or other cell without a cancer-associated genotype or phenotype. Telomerase expression may be increased relative to a control cell, such as a Giant Cell not expressing biomarkers associated with cancer, such as the antigens described herein, or other normal, non-cancerous cells, like a neutrophils, by 1, 2, 5, 10, 20, 30, 40, 50, 60, 70, 80, 90, 100, 150, 200% or more (or any significant difference) based on molecular quantification, including copy number assessment or semiquantitative assessment by imaging and immune-mediated binding (specific antibodies to telomerase or telomerase subunits bound to chromogens or fluorochromes), on weight or molar percentage of telomerase.

In some embodiments, the cancer-associated phenotype or genotype comprises increased levels of mRNA encoding a telomerase or a subunit thereof, or increased expression of a nucleic acid component of telomerase, compared to that of a Giant Cell without a cancer-associated phenotype or genotype or compared to a non-cancerous cell. Such increases in mRNA may range from 1, 2, 5, 10, 20, 30, 40, 50, 60, 70, 80, 90, 100, 150, 200% or more (or any significant difference) based on copy number counting or arbitrary units assessment or REAPseq (RNA expression and protein sequencing assay) (Peterson V M et al. Multiplexed quantification of proteins and transcripts in single cells. NATURE BIOTECHNOLOGY 35:936, 2017) or molar percentages of the mRNA.

In other embodiments, the cancer-associated genotypes or phenotypes comprise over-expression of telomerase or a protein or nucleic acid subunit thereof, compared to that of a Giant Cell without a cancer-associated phenotype or genotype or compared to a non-cancerous cell.

The methods described above may further comprise detecting mutations, epigenetic modifications, or both, in nucleic acids of a Giant Cell.

In some embodiments of these methods, the Giant Cells are isolated from the biological sample using one or more of (i) size exclusion methodology, (ii) an immune-mediated analyte capture element, (iii) red blood cell lysis, and/or (iv) white blood cell depletion/elimination and/or (v) density-based isolation.

In some embodiments, the Giant Cells are isolated by Isolation by SizE of Tumor cells (ISET).

In some embodiments, the Giant Cells are isolated from buffy coat cells.

In certain embodiments, the Giant Cells are isolated by binding to an antibody that recognizes cell surface or intracellular markers of the Giant Cells. These include epitopes of a macrophage antigen, an epithelial antigen, a syncytin, MC1-R, CD163, MAC 87 or DAP12, a prostate antigen; or epitopes of telomerase or its component molecules.

In such methods the analyzing may be performed on a single Giant Cell, or a group of two, three, four, five or more Giant Cells or a population of Giant Cells.

In some embodiments of this method the subject has been diagnosed with cancer; the subject is at risk of cancer but has not been diagnosed with cancer; the subject is being treated for cancer and is being monitored for progression or regression of the cancer; or the subject has cancer in remission and is being monitored for relapse of the cancer.

These methods may further comprise recovering Giant Cells having a cancer-associated genotype or phenotype, for example, by separation from other components of a biological sample, by filtration, affinity purification, or mechanically, such as by centrifugation.

Another aspect of this technology is directed to Giant Cells recovered by the methods disclosed herein, which may, optionally, be substantially free of Giant Cells not having a phenotype or genotype associated with cancer.

Another aspect of this technology is directed to use of the Giant Cells, which may or may not be associated with a cancer genotype or phenotype, recovered by the methods disclosed herein for medical treatment for a subject at risk or having, or having, cancer; or the use of such Giant Cells for diagnostic or laboratory procedures, such as in vitro or ex vivo uses.

In some embodiments, the methods disclosed herein further comprise treating a subject for cancer or administering prophylaxis against cancer to a subject when Cancer Giant Cells are detected in the sample. Such cancers associated with a Cancer Giant Cell phenotype or genotype may be any type of solid or liquid cancer including breast, prostate, pancreatic, NSCLC, sarcoma, kidney, bladder, colon, colorectal, uterine cancer, sarcoma, neuroblastoma, esophageal, ovarian, melanoma, liver, lung cancer, leukemia, lymphoma, Myelodysplastic Syndrome. When the methods disclosed herein further comprise treatment of a cancer associated with a Cancer Giant Cell genotype or phenotype, one skilled in the immunological or oncological arts may select a drug, radiological, or other cancer treatment.

In some embodiments, the treating comprises administering an agent that inhibits the expression of a cancer associated antigen like syncytins, syncytins receptors, HERV proteins and their receptors, CD71, MC1-R, CD163, MAC 87 or DAP12 or other antigens disclosed herein; or which targets or reduces the levels of said antigens in the Giant Cells of the subject; wherein said agent may be an antibody that binds to said antigen or protein, or may be an oligo, miRNA, siRNA or other nucleic acid agent that inhibits the expression or function of said antigens or proteins.

In other embodiments, the treating comprises administering an agent that decreases the levels of telomerase or its components in the Giant Cells of the subject or decreases the expression of mRNA encoding said components.

In some embodiments, the subject is treated by administering Imetelstat, an oligo that interferes with telomerase expression or function, or other telomerase-specific antisense oligonucleotides, miRNA or siRNA that interfere with telomerase expression or function.

Another aspect of this technology is directed to a method of assaying for cancer-associated phenotypic or genotypic features in Giant Cells, said method comprising, consisting essentially of, or consisting of subjecting a biological sample obtained from a subject to a Giant Cells isolation methodology, wherein cells measuring 15, 20, 25, 30, 35, or preferably 40 microns or larger (or other sizes described herein) are collected, wherein the collected cells include Giant Cells and analyzing the collected cells for cancer-associated molecular changes, thereby analyzing cancer-associated molecular changes in Giant Cells.

In this method, the cancer-associated phenotype or genotype may involve expression of at least one macrophage antigen, or at least one epithelial antigen, or a syncytin, or MC1-R, CD 163, MAC 87 or DAP12.

In some embodiments, the cancer-associated phenotype or genotype comprises increased expression of telomerase or a subunit thereof compared to a Giant Cell without a cancer-associated genotype or phenotype.

These methods may further comprise making a prognosis based on the identity of the cancer-associated genotypic or phenotypic features of the Giant Cells; or may further comprise making a prediction of treatment response based on the identity of the genotypic or phenotype features of the Giant Cells; may further comprise diagnosing the subject as having cancer based on the identity of the genotypic or phenotype features of the Giant Cells; or may further comprise diagnosing cancer recurrence in the subject based on the identity of the genotypic or phenotype features of the Giant Cells.

In some embodiments, the methods disclosed above may further comprise Giant Cells having a cancer associated phenotype or genotype or recovering Giant Cells not having a cancer associated genotype or phenotype.

Another aspect of this technology is directed to Giant Cells recovered by the method described above, optionally substantially free of Giant Cells not having a phenotype or genotype associated with cancer. Alternatively, Giant Cells may be recovered by the method described above which lack a cancer-associated genotype or phenotype.

A further aspect of this technology is the use of the Giant Cells recovered by the method described above for medical treatment for a subject at risk or having, or having, cancer; or the use of such Giant Cells for diagnostic or laboratory procedures, such as in vitro or ex vivo uses.

Another aspect of this technology is directed to a method for determining efficacy of a cancer treatment comprising, consisting essentially of, or consisting of isolating Giant Cells from a biological sample of a subject before and after a cancer treatment, and determining a number of Giant Cells having a cancer-associated phenotype or genotype before and after said treatment; wherein an increase in a number of Giant Cells having a cancer-associated phenotype indicates treatment failure and wherein a decrease in a number of Giant Cells having a cancer-associated phenotype indicates efficacy of the treatment.

In some embodiments of this method, said cancer-associated phenotype or genotype comprises presence or level of at least one macrophage antigen, at least one epithelial antigen, a syncytin, HERV proteins, MC1-R, CD 163, MAC 87 or DAP12; or levels of mRNA encoding said antigens or proteins.

In other embodiments of this method said cancer-associated phenotype or genotype comprises increased levels telomerase or a subunit thereof compared to a Giant Cell without a cancer-associated genotype or phenotype; or increased levels of mRNA or encoding said telomerase or subunits thereof compared to a Giant Cell or other cell without a cancer-associated genotype or phenotype.

In some embodiments, the Giant Cells are isolated from the biological sample using one or more of (i) size exclusion methodology, (ii) an analyte capture element, (iii) red blood cell lysis, (iv) white blood cell depletion, or (v) ISET.

In certain embodiments, the Giant Cells are identified or isolated by binding to an antibody that recognizes cell surface or intracellular markers of the Giant Cells; wherein said antibody binds to a macrophage antigen, an epithelial antigen, a syncytin, HERV proteins, MC1-R, CD 163, MAC 87 or DAP12; or by an antibody that binds to telomerase or a component thereof.

In the method disclosed above, a subject may have a cancer selected from the group consisting of breast, prostate, pancreatic, head & neck, NSCLC, sarcoma, kidney, bladder, colon, colorectal, uterine sarcoma, neuroblastoma, esophageal, ovarian, melanoma, liver, and lung cancer. However, other types of solid or liquid cancers associated with the presence of Giant Cells are not excluded.

Another embodiment is directed to method for preventing or treating cancer comprising isolating Tumor Cells/Cancer Giant Cells from a patient or identifying TC/CGC cells in a patient, identifying one or more drugs that kill the cancer cells, and administering a drug regiment comprising said one or more anti-cancer drugs to the patient in an amount sufficient to eliminate the cancer cells in the patient or administering radiation in an amount sufficient to eliminate the giant cancer cells. This method may further comprise treating the patient with a different drug regimen after the elimination of the Tumor Cells/Cancer Giant Cells. In some embodiments, the different drug regimen comprises administering a different drug or drug combination, administering a lower amount of at least one of the anti-cancer drugs than that currently used; or administering a lower amount of radiation than that currently used, to eliminate the Tumor Cells/Cancer Giant Cells. Additional embodiments are listed below.
1. A method for identifying Cancer Giant Cells or circulating Cancer Giant Cells and distinguishing them from Cancer Giant Cells or from circulating non-cancer Giant Cells by phenotype and/or genotype, said method comprising:
   obtaining Giant Cells from a biological liquid or other sample from a subject, and analyzing the Giant Cells for the expression of one or more of the following markers: telomerase or its subunit(s), syncytin, HERV proteins, other tumor markers or aneuploidy thereby identifying Cancer Giant Cells or circulating Cancer Giant Cells having a cancer-associated phenotype and/or genotype,
   wherein said Giant Cells have a major axis diameter of at least 15 microns; or
   a method of identifying Cancer Giant Cells or circulating Cancer Giant Cells by phenotypic markers and developing a personalized, anti-Cancer Giant Cells targeted treatment based on the circulating Cancer Giant Cells markers or on Cancer Giant Cells markers, said method comprising:
(a) isolating Giant Cells having a major axis diameter of at least 15, 20, 25, 30, 35 or 40 microns from a biological liquid or other sample of a subject,
(b) identifying one or more of the following tumor markers in the isolated Giant Cells: telomerase or its subunits, syncytins, HERV proteins, other tumor markers thereby identifying Cancer Giant Cells having a Cancer-associated phenotype or genotype or circulating Cancer Giant Cells having a cancer-associated phenotype or genotype,
(c) developing a personalized targeted treatment against circulating Cancer Giant Cells or Cancer Giant Cells, and,
(d) using the circulating Cancer Giant Cells and Cancer Giant Cells markers as a companion diagnostic test to follow the targeted anti-Cancer Giant Cells treatment.
2. The method of embodiment 1, wherein the cancer-associated phenotype and/or genotype comprises expression of racemase, PSMA, AFP, CD71, syncytins receptors, HER V protein receptors or another tumor marker.
3. The method of embodiment 1 or 2, wherein the cancer-associated phenotype and/or genotype comprises expression of at least one macrophage antigen, at least one epithelial antigen, a syncytin 1 or 2 antigen, MC1-R, CD 163, MAC 87 or DAP12.
4. The method of embodiment 1, 2 or 3, wherein the cancer-associated phenotype and/or genotype comprises increased expression of telomerase or a subunit thereof compared to a non-cancer Giant Cell
5. The method of embodiment 1, 2, 3, or 4, wherein the cancer-associated genotype comprises increased expression or abnormal expression of at least one mRNA encoding a macrophage antigen, and/or encoding epithelial antigen, and/or encoding syncytin, and/or encoding MC1-R, CD163, MAC 87 or DAP12.
6. The method of embodiment 1, 2, 3, 4, or 5, wherein the cancer-associated phenotype and/or genotype comprises increased or abnormal levels of mRNA encoding a telomerase or a subunit thereof, or increased expression or abnormal expression of a nucleic acid component of telomerase, compared to that of a Giant Cell without a cancer-associated phenotype or genotype or compared to a non-cancerous control cell.
7. The method of embodiment 1, 2, 3, 4, 5, or 6, wherein cancer-associated phenotype and/or genotype comprises over-expression of telomerase or a protein or nucleic acid subunit thereof, compared to that of a Giant Cell without a cancer-associated phenotype or genotype or compared to a non-cancerous control cell
8. The method of any one of embodiments 1-7, wherein the cancer-associated genotype or phenotype comprises detecting aneuploidy, abnormal copy number variation (CNV), mutations, epigenetic, methylation modifications, or both, in nucleic acids of a Cancer Giant Cell or Cancer Giant Cells, or wherein said method further comprises detecting aneuploidy, abnormal copy number variation (CNV), mutations, epigenetic, methylation modifications, or both, in nucleic acids of a Cancer Giant Cell or Cancer Giant Cells.
9. The method of any one of embodiments 1-8, wherein the Giant Cells are isolated from the biological liquid or sample using one or more of (i) size exclusion methodology, (ii) an analyte capture element, (iii) red blood cell lysis, (iv) white blood cell depletion/elimination, (v) density-based isolation, (vi) filtration.
10. The method of any one of embodiments 1-9, wherein the Giant Cells are isolated by Isolation by SizE of Tumor cells (ISET).
11. The method of any one of embodiments 1-10, wherein the Giant Cells are isolated from buffy coat cells.
12. The method of any one of embodiments 1-11, wherein the Giant Cells are isolated by binding them to an antibody or ligand that recognizes a cell surface or intracellular marker or determinant of the Giant Cells or Cancer Giant Cells.
13. The method of any one of embodiments 1-12, wherein the analyzing is performed on a single Giant Cell, or a group of two or more Giant Cells.
14. The method of any one of embodiments 1-13, wherein the subject is at risk of cancer but has not been diagnosed with cancer.
15. The method of any one of embodiments 1-13, wherein the subject has been diagnosed with cancer.
16. The method of any one of embodiments 1-13, wherein the subject is being treated for cancer and/or is being monitored for progression or regression of the cancer.
17. The method of any one of embodiments 1-13, wherein the subject has cancer in remission and is being monitored for relapse of the cancer.
18. The method of any one of embodiments 1-17, further comprising recovering Giant Cells having a cancer-associated phenotype or genotype or recovering Circulating Giant Cancer Cells.
19. Giant Cells recovered by the method of embodiment 18, optionally, substantially free of Giant Cells not having a phenotype or genotype associated with cancer; or optionally substantially free of one or more types of other hematological or somatic cells.
20. Use of the Giant Cells, Cancer Giant Cells, or Circulating Giant Cancer Cells recovered by the method of embodiment 18 as a medicament or medical treatment for a subject at risk or having, or having, cancer.
21. The method of any one of embodiments 1-20, further comprising treating a subject for cancer or administering prophylaxis against cancer to a subject when Cancer Giant Cells or Circulating Cancer Giant Cells are detected in the sample.
22. The method of embodiment 21, wherein the cancer is selected from the group consisting of breast, prostate, pancreatic, NSCLC, sarcoma, kidney, bladder, colon, colorectal, uterine sarcoma, neuroblastoma, esophageal, ovarian, melanoma, liver, lung cancer, lymphoma, and leukemia; or any other type of solid or liquid cancer.
23. The method of embodiment 21 or 22, wherein the treating comprises administering an agent that targets Cancer Giant Cells; wherein said agent targets telomerase or its subunit(s) and/or antigens and/or proteins expressed by Cancer Giant Cells; wherein said agent targets the expression of one or more of: a macrophage antigen, an epithelial antigen, a syncytin, AFP, racemase, PSMA, CD71, MC1-R, CD163, MAC 87, DAP12;
wherein said agent eliminates or reduces the levels of Cancer Giant Cells of the subject; wherein said agent comprises an antibody or ligand that binds to a circulating Cancer Giant Cell or Cancer Giant Cell antigen or protein; wherein said agent comprises a bi-specific antibody that binds to two Cancer Giant Cell antigens or proteins; wherein said agent comprises a BiTE bi-specific or tri-specific antibody with binding specificity for an activating receptor expressed by T cells (usually CD3); wherein such agent is an antibodies-drug conjugate; wherein such agent is a nanotherapy, such as a vectorized CGC-targeted nanoparticles delivering drug; wherein such agent is a CGC-targeted epigenetic drug: wherein said agent is an anticancer drug that targets Cancer Giant Cells; or wherein said agent comprises an oligo, mRNA, miRNA, siRNA or other nucleic acid agent that inhibits the expression or function of said Cancer Giant Cell antigens or proteins; wherein the agent, such as mRNA encoding a cancer antigen, induces an immune response against Cancer Giant Cell antigens; or wherein the agent comprises Imetelstat (which is an oligo that interferes with telomerase function), comprises telomerase-specific antisense oligonucleotides, or comprises antibodies or antibody conjugates that bind to macrophage-specific markers, such as CD163 (depletion of CD163 has been associated with reduced tumor growth and progression) and/or to telomerase peptides.
24. The method of embodiment 21, 22, or 23, wherein the treating comprises administering an agent that decreases the levels of telomerase or its subunits(s) or components in the circulating Cancer Giant Cells or the Cancer Giant Cells of the subject or decreases the expression of mRNA encoding said components.
25. The method of embodiment 21, 22, 23, or 24 wherein the subject is treated by administering Imetelstat, an oligo that interferes with telomerase expression or function, or other telomerase-specific antisense oligonucleotides, miRNA or siRNA that interfere with telomerase expression or function.
26. A method of assaying for cancer-associated phenotypic or genotypic features in Giant Cells, said method comprising:
   subjecting a biological liquid or other sample obtained from a subject to a size exclusion methodology, wherein cells measuring 40 microns or larger are collected, wherein the collected cells include Giant Cells and
   analyzing the collected cells for cancer-associated molecular changes, thereby analyzing cancer-associated molecular changes in Giant Cells.
27. The method of embodiment 26, wherein the cancer-associated phenotype or genotype comprises expression of at least one macrophage antigen, at least one epithelial antigen, a syncytin, a HERV protein, CD71, AFP, Racemase, PSMA, MC1-R, CD 163, MAC 87 or DAP12.
28. The method of embodiment 26 or 27, wherein the cancer-associated phenotype or genotype of a circulating Cancer Giant Cell or Cancer Giant Cell comprises increased expression of telomerase or a subunit thereof compared to a Giant Cell without a cancer-associated phenotype or genotype.
29. The method of embodiment 26, 27, or 28, further comprising making a prognosis based on the identity of the cancer-associated phenotypic or genotypic features of the Giant Cells, Cancer Giant Cells, or circulating Cancer Giant Cells.
30. The method of embodiment 26, 27, 29, or 29, further comprising making a prediction of treatment response based on the identity of the phenotypic or genotype features of the Giant Cells, Cancer Giant Cells, or circulating Cancer Giant Cells.
31. The method of any one of embodiments 26-30, further comprising diagnosing the subject as having cancer based on the identity of the phenotypic or genotype features of the Giant Cells, Cancer Giant Cells, or circulating Cancer Giant Cells . . .
32. The method of any one of embodiments 26-31, further comprising diagnosing cancer recurrence in the subject based on the identity of the phenotypic or genotype features of the Cells, Cancer Giant Cells, or circulating Cancer Giant Cells.
33. The method of any one of embodiments 26-32, further comprising recovering Giant Cells, Giant Cancer Cells, or circulating Cancer Giant Cells having a cancer associated phenotype or genotype.
34. Giant Cells recovered by the method of embodiment 33, optionally substantially free of Giant Cells not having a phenotype or genotype associated with cancer.
35. Use of the Giant Cells recovered by the method of embodiment 34 as a medicament or medical treatment for a subject at risk or having, or having, cancer.
36. A method for determining efficacy of a cancer treatment comprising:
   isolating Giant Cells from a biological sample of a subject before and after a cancer treatment, and determining a number of Giant Cells having a cancer-associated phenotype or genotype before and after said treatment; wherein an increase in a number of Giant Cells having a cancer-associated phenotype indicates treatment failure and wherein a decrease in a number of Giant Cells having a cancer-associated phenotype indicates efficacy of the treatment.
37. The method of embodiment 36, wherein said cancer-associated phenotype or genotype comprises presence or level of at least one macrophage antigen, at least one epithelial antigen, a syncytin, a HERV protein, CD71, AFP, PSMA, racemase, MC1-R, CD 163, MAC 87 or DAP12; or levels of mRNA encoding said antigens or proteins.
38. The method of embodiment 36 or 37, wherein said cancer-associated phenotype or genotype comprises increased levels of telomerase or a subunit thereof compared to a Giant Cell without a cancer-associated phenotype or genotype; or increased levels of mRNA or encoding said telomerase or subunits thereof compared to a Giant Cell or other cell without a cancer-associated phenotype or genotype.
39. The method of embodiment 36, 37, or 38, wherein the Giant Cells are isolated from the biological liquid or sample using one or more of (i) size exclusion methodology, (ii) an analyte capture element, (iii) red blood cell lysis, (iv) white blood cell depletion, (v) a density-based isolation, or (vi) ISET.
40. The method of embodiment 36, 37, 38, or 39, wherein the size exclusion methodology comprises use of a filter that retains cells of 40 microns or larger.
41. The method of embodiment 36, 37, 38, 39, or 40, wherein the Giant Cells are identified or isolated by binding to an antibody that recognizes cell surface or intracellular markers of the Giant Cells; wherein said antibody binds to a telomerase peptide, a macrophage antigen, an epithelial antigen, a syncytin, a HERV protein, MC1-R, CD 163, MAC 87 or DAP12.
42. The method of any one of embodiments 36-41, wherein the subject has a cancer selected from the group consisting of breast, prostate, pancreatic, NSCLC, sarcoma, kidney, bladder, colon, colorectal, uterine sarcoma, neuroblastoma, esophageal, ovarian, melanoma, liver, and lung cancer.
43. A method for preventing or treating cancer comprising isolating Cancer Giant Cells from a patient or identifying Cancer Giant Cells or circulating Cancer Giant Cells in a patient, identifying one or more drugs that kill the Cancer Giant Cells or circulating Cancer Giant Cells, and administering a drug regiment comprising said one or more anti-cancer drugs to the patient in an amount sufficient to eliminate the cancer giant cells in the patient or administering radiation in an amount sufficient to eliminate the cancer Giant Cells or circulating Cancer Giant Cells.
44. A kit for detecting Cancer Giant Cells or circulating Cancer Giant Cells, to be used for early cancer and early cancer recurrence detection, management of cancer treatment, determination of therapy and of treatment response is disclosed, comprising at least one antibody or other ligand that binds to a cancer marker on a Cancer Giant cell or circulating Cancer Giant Cells, or at least one probe or primer that can bind to or amplify a biomarker on a Cancer Giant Cell or circulating Cancer Giant Cells and optionally containers for said antibodies or ligands, for said probes or primers, reagents for detection of said biomarkers, instructions for use, or other components, supplies or equipment for detecting said biomarkers.

Other terminology. Unless expressly stated, the terms used herein are intended to have the plain and ordinary meaning as understood by those of ordinary skill in the art. The following definitions are intended to aid the reader in understanding the present disclosure but are not intended to vary or otherwise limit the meaning of such terms unless specifically indicated.

It should be noted that, as used in the specification and the claims, the singular forms “a,” “an,” and “the” include plural referents unless the context clearly dictates otherwise.

As used herein, the term “and/or” includes any and all combinations of one or more of the associated listed items and may be abbreviated as “/”. A and/or B includes A, B, and (A+B).

As used herein in the specification, including as used in the examples and unless otherwise expressly specified, all numbers may be read as if prefaced by the word “substantially”, “about” or “approximately,” even if the term does not expressly appear. The phrase “about” or “approximately” may be used when describing magnitude and/or position to indicate that the value and/or position described is within a reasonable expected range of values and/or positions.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present invention that do not contain those elements or features.

All publications and patent applications mentioned in this specification are herein incorporated by reference in their entirety to the same extent as if each individual publication or patent application was specifically and individually indicated to be incorporated by reference, especially referenced is disclosure appearing in the same sentence, paragraph, page or section of the specification in which the incorporation by reference appears.

The citation of references herein does not constitute an admission that those references are prior art or have any relevance to the patentability of the technology disclosed herein. Any discussion of the content of references cited is intended merely to provide a general summary of assertions made by the authors of the references and does not constitute an admission as to the accuracy of the content of such references.

1. A method of early cancer detection comprising:
a. screening a subject for cancer, comprising detecting circulating Cancer Giant Cells in a biological liquid from a subject previously determined to be at risk for cancer, or at risk for cancer recurrence, wherein said detecting comprises:
(aa) isolating Giant Cells that have a major axis diameter of at least 15, 20, 25, 30, 35, 40 microns from a biological liquid of a subject;
(bb) detecting telomerase or its subunit(s) or at least one other cancer cell phenotype or marker in the isolated Giant Cells from step (aa), thereby identifying Cancer Giant Cells;
b. optionally treating the subject with a therapy to eliminate the cancer detected using the Cancer Giant Cells biomarker identified in step (bb).

2. The method of claim 1, wherein the subject has not been previously diagnosed with cancer.

3. The method of claim 1, wherein the subject has been diagnosed with cancer, is in remission from cancer, or is being monitored for progression or regression of cancer.

4. The method of claim 1, wherein the biological liquid is blood.

5. The method of claim 1, wherein the biological liquid is urine.

6. The method of claim 1, wherein the biological liquid is saliva.

7. The method of claim 1, wherein the biological liquid comprises sperm, prostatic fluid, or seminal fluid.

8. The method of claim 1, wherein the cancer cell phenotype comprises expression of at least one of the following markers: telomerase or its subunit(s), macrophage antigen, epithelial antigen, a syncytin, a HERV protein, CD71, AFP, Racemase, PSMA, MC1-R, CD 163, MAC 87 or DAP12.

9. The method of claim 1, wherein the cancer cell phenotype comprises aneuploidy, abnormal copy number variation (CNV), mutations, or epigenetic modifications compared to a non-cancerous control cell.

10. The method of claim 1, wherein in step (bb) the at least one cancer phenotype comprises increased or abnormal expression of telomerase or subunit(s) thereof.

11. The method of claim 1, wherein in step (bb) the at least one cancer cell phenotype comprises an increased expression of a syncytin 1 or syncytin 2 compared to a non-cancerous control cell.

12. The method of claim 1, wherein in step (bb) the at least one cancer phenotype comprises increased expression of a syncytin; and said method further comprises performing the optional treating in step (b) by administering a drug or biologic that targets syncytin.

13. The method of claim 1, wherein the subject has not been previously diagnosed with cancer, and wherein the detection of the Cancer Giant Cells in step (bb) allows cancer treatment in a timely manner that targets cancer cells expressing the cancer cell phenotype.

14. The method of claim 1, wherein the subject has not been previously diagnosed with cancer recurrence and wherein the detection of Cancer Giant Cells in step (bb) allows a timely personalized cancer treatment that targets cancer cells expressing the cancer cell phenotype.

15. The method of claim 1, wherein the subject is being monitored for progression or regression of cancer and the Cancer Giant Cells biomarker identified in step (bb) permits timely cancer treatment of progressing or regressing cancer in the subject that targets cancer cells expressing the cancer cell phenotype.

16. The method of claim 1 for treatment of prostate cancer in the subject, wherein in step (a) the biological sample is urine, wherein in step (bb) the at least one cancer cell phenotype comprises an increased level of a least one of the following markers: telomerase or its subunit(s), PSMA, syncytin, racemase compared to a non-cancerous control cell.

17. The method of claim 16, wherein in step (bb) the at least one cancer phenotype comprises increased expression of a telomerase and wherein the method further comprises treatment step (b) comprising administering a drug, biologic, agent, or immunotherapy that targets telomerase or its subunits.

18. The method of claim 16, wherein in step (bb) the at least one cancer phenotype comprises increased expression of a syncytin, wherein said method further comprises step (b) comprising administering a drug, biological, agent, or immunotherapy that targets syncytin.

19. The method of claim 1 further comprising detecting Circulating Tumor Cells (CTCs) in the biological liquid.

20. A kit for detecting Cancer Giant Cells comprising at least one antibody or other ligand that binds to a cancer cell phenotype or marker on a Cancer Giant cell; at least one probe, primer, or set of probes or primers that binds to or amplifies a biomarker of a Cancer Giant Cell; and optionally, containers for said antibodies or ligands or for said probes or primers, reagents for detection of said biomarkers; supplies or equipment for detecting said biomarker(s); and optionally instructions for use of the kit for early cancer detection, early cancer recurrence detection, management of cancer treatment, determination of efficacy of cancer detection, monitoring or determining efficacy of a cancer treatment.

21. A method of early cancer detection comprising screening a subject for cancer, comprising detecting circulating Cancer Giant Cells in a biological liquid from a subject previously determined to be at risk for cancer, or at risk for cancer recurrence, wherein said detecting comprises: (aa) isolating Giant Cells that have a major axis diameter of at least 15 microns from a biological liquid of a subject; (bb) detecting telomerase or its subunit(s) or at least one cancer cell phenotype in the isolated circulating Cancer Giant Cells from step (aa), thereby identifying circulating Cancer Giant Cells.

22. The method of claim 21, further comprising treating the subject for a type of cancer expressing the cancer cell phenotype identified by step (bb).
